(19) United States
(12) Patent Application Publication
(10) Pub. No.: US 2024/0341310 A1
(43) Pub. Date: Oct. 17, 2024

(54) ANTIPATHOGENIC NANOSTRUCTURES

(71) Applicant: THE BOEING COMPANY, Arlington, VA (US)

(72) Inventors: Michael J. MONTEIRO, Brisbane (AU); Valentin A. BOBRIN, Brisbane (AU)

(21) Appl. No.: 18/294,426

(22) PCT Filed: Aug. 3, 2022
(86) PCT No.: PCT/US2022/074472

§ 371 Date: Feb. 1, 2024

Related U.S. Application Data
(60) Provisional application No. 63/341,347, filed on May 12, 2022.
(60) Provisional application No. 63/299,723, filed on Jan. 14, 2022.
(60) Provisional application No. 63/228,963, filed on Aug. 3, 2021.

(51) Int. Cl. A01N 47/44; A01N 25/34; A01P 1/00; A61L 2/232; A61L 101/32; A61L 101/40; A61L 101/44; A61L 101/50; A62B 18/08; B82Y 5/00

(52) U.S. Cl. CPC A01N 47/44; A01N 25/34; A01P 1/00; A61L 2/232; B82Y 5/00; A61L 2101/32; A61L 2101/40; A61L 2101/44; A61L 2101/50; A62B 18/08

(57) ABSTRACT

Methods include disposing a layer of a solution or emulsion having a nanostructure on a surface. The surface further includes a SARS-CoV-2 virus disposed thereon. The nanostructure includes a compound or salt thereof. The compound includes one or more styrene units, one or more N-alkylacrylamide units, and a moiety represented by the formula:

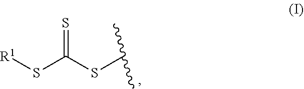

where R¹ is alkyl. The compound includes a moiety represented by the formula:

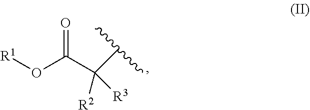

where R¹ is alkyl and R² and R³ are independently hydrogen or alkyl. The compound includes a plurality of N,N-(dialkylamino)(divalent alkyl) alkylacrylate units. One or more of the N,N-(dialkylamino)(divalent alkyl)alkylacrylate units has an unsubstituted nitrogen. One or more of the N,N-(dialkylamino)(divalent alkyl)alkylacrylate units is substituted with a C₁-C₁₆ alkyl moiety, and one or more of the N,N-(dialkylamino)(divalent alkyl) alkylacrylate units is substituted with a moiety selected from the group consisting of guanidine, polygalactose, coumarin, and combination(s) thereof.

### CROSS-REFERENCE TO RELATED APPLICATIONS

This application is a PCT application claiming priority to U.S. provisional application Ser. No. 63/228,963, filed Aug. 3, 2021, U.S. provisional patent application Ser. No. 63/299,723, filed Jan. 14, 2022, and U.S. provisional application Ser. No. 63/341,347, filed May 12, 2022, each of which are incorporated herein by reference in their entirety.

### FIELD

The present disclosure provides an antipathogenic nanostructures, such as a coating of antipathogenic nanostructures over a surface of a vehicle, a building, a wearable, a filter, or any other suitable object.

### BACKGROUND

Pandemics by pathogens have major and lingering impacts due to loss of immunity over time from either vaccination or previous infection. High touch surfaces and enclosed environments including vehicles, offices, transportation facilities, habitation, among others are concerned with preventing the transmission of pathogens, such as viruses and microbes.

Airline passengers are concerned with aircraft cabin sterility and space transportation, and habitation industries are concerned with preventing the transmission of pathogens, such as viruses and microbes. Travelers in space may become more easily immunosuppressed with a greater susceptibility to disease transmission by pathogens. In some cases, microbes may replicate more and become more virulent in a zero gravity environment or a radiation shielded environment.

Preventing disease transmission on aircrafts and spacecrafts has conventionally focused on improvements of the air filtration systems, such as HEPA air filter systems. Replacing and maintaining HEPA filters may be costly or impractical, such as replacing and maintaining HEPA filters. Moreover, such systems may be ineffective to reduce or stop the transmission of pathogens from surfaces. Bacteria and viruses can linger on surfaces for days and even up to a week.

Therefore, there is a need for antipathogenic surface coatings that are effective on reducing the transmission of pathogens, such as microbes and viruses.

### SUMMARY

The present disclosure provides a method of disposing a nanostructure onto a surface. The method includes disposing a layer of a solution or emulsion comprising the nanostructure on the surface. The surface includes a SARS-CoV-2 virus disposed thereon. The nanostructure includes a compound or salt thereof. The compound includes one or more styrene units; one or more N-alkylacrylamide units; a moiety represented by the formula:

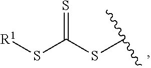

wherein R¹ is alkyl. The compound includes a moiety represented by the formula:

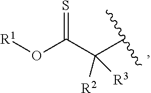

wherein R¹ is alkyl and R² and R³ are independently hydrogen or alkyl. The compound includes a plurality of N,N-(dialkylamino)(divalent alkyl) alkylacrylate units. One or more of the N,N-(dialkylamino)(divalent alkyl)alkylacrylate units has an unsubstituted nitrogen. One or more of the N,N-(dialkylamino)(divalent alkyl)alkylacrylate units is substituted with an octane moiety. One or more of the N,N-(dialkylamino)(divalent alkyl) alkylacrylate units is substituted with a moiety selected from the group consisting of guanidine, polygalactose, coumarin, and combination(s) thereof.

In some aspects, a method of disposing a nanostructure onto a surface includes disposing the nanostructure on the surface. The nanostructure includes a compound, and the compound includes one or more N-isopropylacrylamide units; a moiety represented by the formula:

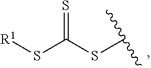

wherein R¹ is alkyl. The compound includes a moiety represented by the formula:

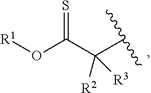

wherein R¹ is alkyl and R² and R³ are independently hydrogen or alkyl. The compound includes a plurality of N,N-(dimethylamino)ethyl methacrylate units. One or more of the N,N-(dimethylamino)ethyl methacrylate units have an unsubstituted nitrogen. One or more of the N,N-(dimethylamino)ethyl methacrylate units is substituted with an octane moiety. One or more of the N,N-(dimethylamino)ethyl methacrylate units is substituted with a moiety selected from the group consisting of guanidine, polygalactose, coumarin, and combination(s) thereof.

In some aspects, a nanostructure includes a compound, or salt thereof, and the compound includes one or more N-isopropylacrylamide units; a moiety represented by the formula:

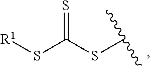

wherein R¹ is alkyl. The compound includes a moiety represented by the formula:

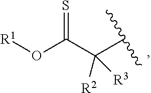

wherein R¹ is alkyl and R² and R³ are independently hydrogen or alkyl. The compound includes a plurality of N,N-(dimethylamino)ethyl methacrylate units. One or more of the N,N-(dimethylamino)ethyl methacrylate units have an unsubstituted nitrogen. One or more of the N,N-(dimethylamino)ethyl methacrylate units is substituted with a C₁-C₁₆ alkyl moiety. One or more of the N,N-(dimethylamino)ethyl methacrylate units is substituted with a moiety selected from the group consisting of guanidine, polygalactose, coumarin, and combination(s) thereof.

### BRIEF DESCRIPTION OF THE DRAWINGS

So that the manner in which the above recited features of the present disclosure can be understood in detail, a more particular description of the disclosure, briefly summarized above, may be had by reference to aspects, some of which are illustrated in the appended drawings. It is to be noted, however, that the appended drawings illustrate only typical aspects of this present disclosure and are therefore not to be considered limiting of its scope, for the present disclosure may admit to other equally effective aspects.

FIG. 1A is a schematic view illustrating a nanoworm according to certain aspects of the present disclosure.

FIG. 1B depicts an illustration of a synthesis scheme of forming a nanostructure according to certain aspects of the present disclosure.

FIG. 1C depicts an illustration of a synthesis scheme of forming a nanostructure according to certain aspects of the present disclosure.

FIG. 1D depicts an illustration of a synthesis scheme of forming a nanostructure according to certain aspects of the present disclosure.

FIG. 2 is a graph illustrating the polymer per surface area with increasing amounts of coatings according to certain aspects of the present disclosure.

FIG. 3 is images illustrating the spreading of water droplet at pH 6.5 on surfaces according to certain aspects of the present disclosure.

FIG. 4 is a graph illustrating the relative increase in droplet spreading with coating number according to certain aspects of the present disclosure.

FIG. 5 depicts a graphical representation of the presence of SARS-CoV-2 (alpha variant) titer by TCID₅₀ according to certain aspects of the present disclosure.

FIG. 6 depicts a graphical representation of the presence of intact SARS-CoV-2 E gene (alpha variant) via quantitative RT-PCR according to certain aspects of the present disclosure.

FIG. 7 depicts the presence of virucidal activity of nanoworm coated surfaces against SARS-CoV-2 delta variant according to certain aspects of the present disclosure.

FIG. 8 depicts the presence of intact SARS-CoV-2 E gene delta variant via quantitative RT-PCR according to certain aspects of the present disclosure.

FIG. 9 depicts a graphical representation of the presence of SARS-CoV-2 omicron variant titer by TCID₅₀ according to certain aspects of the present disclosure.

FIG. 10 depicts a graphical representation of the presence of intact SARS-CoV-2 E gene omicron variant via quantitative RT-PCR according to certain aspects of the present disclosure.

### DETAILED DESCRIPTION

The rise in coronavirus variants has resulted in surges of the disease across the globe. The mutations in the spike protein on the surface of the virion membrane not only allow for greater transmission but raise concerns about vaccine effectiveness. Preventing the spread of SARS-CoV-2, variants of SARS-CoV-2 and other viruses from person-to-person via airborne or surface transmission requires effective inactivation of the virus.

The present disclosure provides a nanostructure, such as a coating of antipathogenic nanoworms over a surface of a vehicle, a building, a wearable, a filter, or any other suitable object. The coating has antipathogenic properties effective at reducing or eliminating pathogens and/or reducing the transmission of pathogens. As used herein, the term “pathogen” refers to viruses, bacteria, fungi, and/or other microbes or germs. The coatings described herein are capable of reducing or eliminating the presence of and/or transmission of a wide range of pathogens, such as SARS-CoV-2 and variants thereof, such as alpha, beta, delta, omicron, or combinations thereof. As used herein, a “nanostructure” refers to a 3-dimensional structure provided by two or more macromolecules (that may have the same or different chemical structure). A “nanoworm” is an example of a nanostructure and has a high aspect ratio (length divided by width). A “nanorod” is an example of a nanostructure that has a low aspect ratio (length divided by width) as compared to a nanoworm.

The coating can be deployed using any suitable method, such as by a water-borne spray-on nanocoating. The spray-on nanocoating can inactivate virion particles and degrade an RNA portion of the virus. Without being bound by theory, it is believed that the nanostructure of the coating binds and, through subsequent large nano-scale conformational changes ruptures the viral membrane. Subsequently, the nanostructure of the coating binds and degrades the RNA of the virus, inactivating the virus, such as SARS-CoV-2 (VIC01) and an evolved B.1.1.7 (alpha) variant, influenza A and a surrogate capsid pseudovirus expressing the influenza A virus attachment glycoprotein, hemagglutinin. The polygalactose functionality on the nanostructure targets the conserved S2 subunit on the SARS-CoV-2 virion surface spike glycoprotein for stronger binding, and the additional attachment of the guanidine groups is known to catalyze the degradation of the RNA genome of the virus.

In some examples, a nanostructure of the present disclosure is coated onto a surface of an item of personal protective equipment, such as a mask, a face shield, a rebreather, a filter cartridge, or combinations thereof. Coating surgical masks with the nanostructures resulted in complete inactivation of VIC01 and B.1.1.7, providing a powerful control measure for SARS-CoV-2 and its variants. Inactivation was further observed for the influenza A and an AAV-HA capsid pseudovirus, providing broad viral inactivation when using a nanoworm of the present disclosure. The technology described herein represents an environmentally friendly coating with a proposed nano-mechanical mechanism for inactivation of viruses both enveloped and capsid. The functional nanostructures can be modified to target other viruses known and unknown, and are compatible with large scale manufacturing processes.

In certain aspects, a nanostructure coated surface is or can become a hydrophilic surface. For example, a nanostructure coated surface is or can become hydrophilic (water soluble) allowing the wetting of a droplet, such as a mucosal drop, blood, urine, sweat, other bodily fluids, and other non-bodily fluids, across the nanostructure coated surface. In certain aspects, pathogens on the surface of the droplet or suspended within the droplet can be captured, inactivated, or deactivated by the nanostructure coated surface. The coatings described herein can include a polymer and may have a transparent appearance when applied to surfaces. In some aspects, the coatings are useful for inactivating one or more, such as all, variants of SARS-CoV-2. Without being bound by theory, it is believed that the coatings target the highly glycosylated spike protein on the virion surface and disrupt the viral membrane through a process of conformational change in the nanoworms to perform a mechanical rupture of the virus membrane.

As used herein, the term “SARS-CoV-2 variant” refers to viruses that have mutated from SARS-CoV-2. The mutations can include about 1 to about 75 mutations across the virus genome, such as about 25 to about 50 mutations. One or more the mutations can include mutations in the spike protein of the virus, such as about 1 to about 40 mutations in the spike protein, such as about 32 mutations. It is believed that certain known variants have enhanced binding to the ACE2 receptor through the receptor binding domain on the spike protein found predominantly on human throat and lung cells. Once bound to the cell, the mutation close to the S1/S2 region further enhances cleavage mainly by the serine proteinase (e.g., TMPRSS2) on the cell surface, exposing the spike's hydrophobic region to fuse and release the viral RNA within the cell, or enhance cell-cell fusion of giant multi-nuclear cells. Different variants can have different responses to vaccination, different rates of transmission, and different symptoms upon contraction. An antigenic shift, due to the high number of mutations in certain variants, such as the omicron spike, may stem from extensive replication in immune-deficient hosts or transmissions back and forth between humans and rodents.

Infected hosts can release SARS-CoV-2 into the environment via sneezing, coughing and skin contact, resulting in potential fomite contamination of surrounding surfaces. Infectious SARS-CoV-2 has been proven in laboratory-based studies to persist on many different surfaces. Personal protective equipment (e.g. a face mask, a face shield, a rebreather, a filter cartridge, or combinations thereof) and treatment of high-touch surfaces with antiviral coatings of the present disclosure can provide long-lasting (e.g., days, weeks, months, etc.) disinfection of contaminated surfaces to reduce or eliminate the spread of SARS-CoV-2.

### Nanostructure Coating

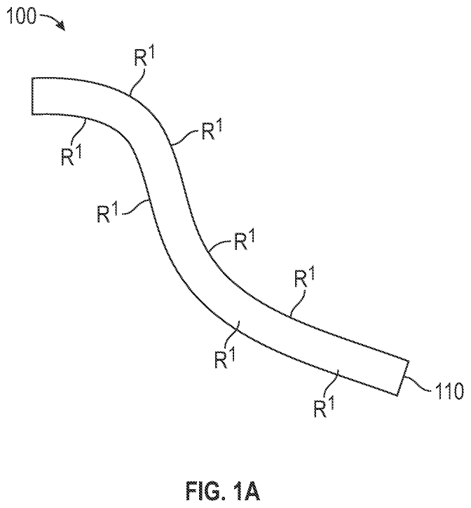

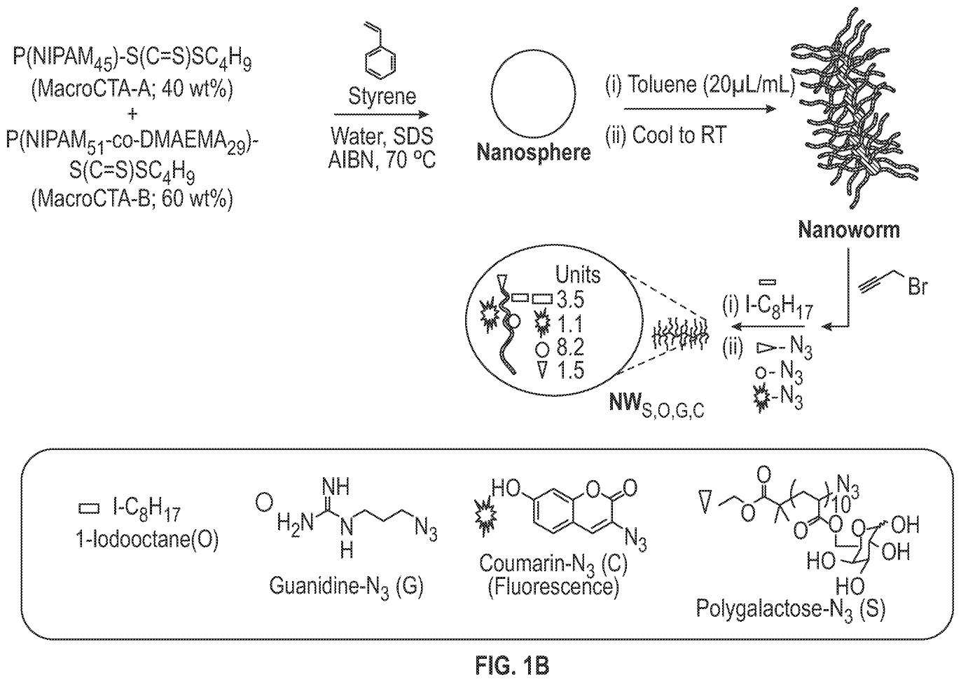

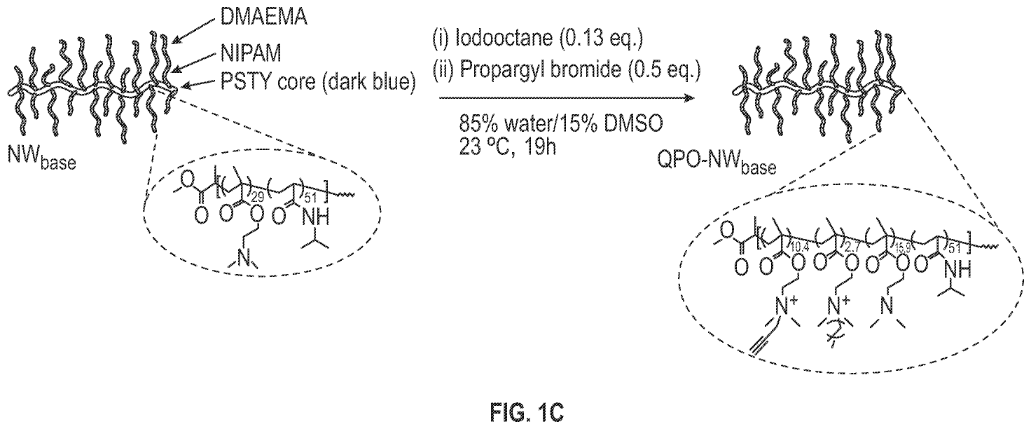

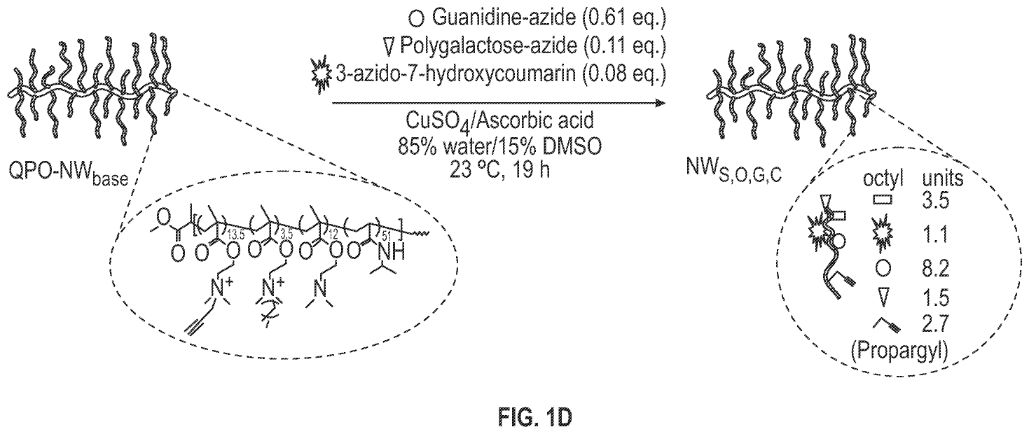

FIG. 1A is a schematic view illustrating a nanostructure, such as a nanoworm 100 according to certain aspects. A backbone or core 110 of the nanoworm 100 includes alkene units and the macroCTA polymer units. The nanoworm 100 includes R¹ groups from the macroCTA polymer units. Each of the R¹ groups is a component from a reversible addition-fragmentation chain-transfer (RAFT) agent, which can be pre-functionalized or post-functionalized. In some examples, each of the R¹ groups is selected to modify the capture and inactivation/deactivation efficiency of the nanoworm 100 and/or to modify the responsiveness (e.g., temperature, pH, salinity concentration, light, and/or combinations thereof) of the nanoworm 100.

The nanostructure described herein includes a compound or salt thereof. The compound includes one or more styrene units, one or more N-alkylacrylamide units, a moiety (as an end cap) represented by the formula:

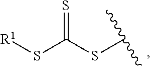

where R¹ is alkyl. The compound further includes a plurality of N,N-(dialkylamino)(divalent alkyl) alkylacrylate units. One or more of the N,N-(dialkylamino)(divalent alkyl)alkylacrylate units has an unsubstituted nitrogen. One or more of the N,N-(dialkylamino)(divalent alkyl)alkylacrylate units is substituted with an octane moiety. One or more of the N,N-(dialkylamino)(divalent alkyl) alkylacrylate units is substituted with a moiety such as guanidine, polygalactose, coumarin, or combination(s) thereof. In some aspects, the nanostructure is a nanoworm, a nanorod, or combinations thereof. The compound further includes a moiety (as an end cap) represented by the formula:

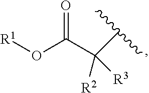

wherein R¹ is alkyl (branched or linear, substituted or unsubstituted) and R² and R³ are independently hydrogen or alkyl (branched or linear, substituted or unsubstituted).

### Three Dimensional Structures of a Compound or Composition

Compounds or compositions of the present disclosure can have a 3-dimensional structure that is a nanoworm or nanorod. A nanorod can have an aspect ratio from about 10:1 to about 1000:1, such as from about 10:1 to about 100:1, such as from about 25:1 to about 75:1. A nanorod can have a diameter from about 10 nm to about 20 nm and a length from about 100 nm to about 10 microns, such as from about 1 micron to about 2 microns. A nanoworm has an aspect ratio of greater than about 1000:1.

Compounds and compositions of the present disclosure can also have a three-dimensional structure that is a sphere, vesicle, donut or lamella sheet. The three-dimensional structure of compositions of the present disclosure can be stable in water for long periods of time (e.g., a nanoworm stable for a year or more at room temperature) and can also be freeze-dried and rehydrated without structural reorganization. For example, a nanoworm solution can be freeze-dried to give dry power. The freeze-dried product can be rehydrated in Milli-Q water at −8 wt % for 2 h. The ability of a composition of the present disclosure to be freeze-dried provides stable transportation of compositions of the present disclosure.

### Synthesis of the Nanoworm

A method of forming a nanostructure is provided. In some aspects, the polymer nanostructures having nanoworm morphology can be produced directly in water using an emulsion polymerization method. The method includes introducing, in a reactor, a styrene monomer with (1) a first polymer having N-alkylacrylamide units and (2) a second polymer having N,N-(dialkylamino)(divalent alkyl)alkylacrylate units and N-alkylacrylamide units to form a mixture. In some aspects, introducing the styrene monomer with the first polymer and the second polymer is performed at a temperature of about −10° C. to about 10° C.

The first polymer consists of the N-alkylacrylamide units as N-isopropylacrylamide units, a moiety represented by the formula:

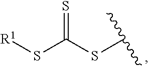

where R¹ is alkyl, and a moiety represented by the formula:

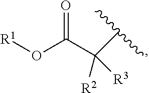

where R¹ is alkyl (branched or linear, substituted or unsubstituted) and R² and R³ are independently hydrogen or alkyl (branched or linear, substituted or unsubstituted). [0037]. In some aspects, the first polymer is free of N,N-(dialkylamino)(divalent alkyl)alkylacrylate units. The second polymer consists of the N,N-(dialkylamino)(divalent alkyl)alkylacrylate units as N,N-(dimethylamino)ethyl methacrylate units, the N-alkylacrylamide units as N-isopropylacrylamide units, a moiety represented by the formula:

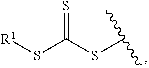

where R¹ is alkyl, and a moiety represented by the formula:

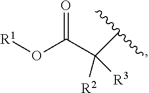

where R¹ is alkyl (branched or linear, substituted or unsubstituted) and R² and R³ are independently hydrogen or alkyl (branched or linear, substituted or unsubstituted).

The method includes introducing an initiator compound to the mixture to form a second mixture having the nanostructure. The initiator compound is a peroxide, a hydroperoxide, or an azo initiator. In some examples, the initiator is azobisisobutyronitrile.

The nanostructures can be coupled with a variety of functional groups, including the hydrophobic octane (O), guanidine (G), a fluorescence probe (coumarin, C) and polygalactose (S) as shown in FIG. 1B. Binding to the highly glycosylated spike S protein was targeted through (i) the strong multivalent binding with polygalactose, and (ii) electrostatic interactions between the negatively charged viral particles and the positively charged guanidine and N,N-(dimethylamino)ethyl methacrylate (DMAEMA) groups. The attached octane groups facilitate the rupture of the viral membrane, in which the viral mRNA can either be degraded by the guanidine groups or electrostatically captured by the polymer coating. The polymer nanostructures described herein can be coated on surfaces, including a surgical mask, and readily inactivated the influenza A virus, ancestral SARS-CoV-2 isolate, alpha variant, and omicron variant.

In certain aspects, the nanostructure includes a copolymer of a macro chain transfer agent (macroCTA) polymer units and alkene units. A macroCTA polymer is a polymer formed by RAFT using a RAFT agent in the polymerization of one or more ethylenically unsaturated monomers.

In some examples, two macro-chain transfer RAFT poly(N-isopropylacrylamide) (PNIPAM) agents were produced from a single non-functional RAFT agent. The emulsion polymerization using the two macro-chain transfer agents in the presence of styrene and initiated by azobisisobutyronitrile (AIBN) at 70° C. in a 500 mL reactor produced spherical particles consisting of two block copolymers of MacroCTAs A and B with polystyrene at an approximately 8 wt % of polymer in water. After the addition of a small amount of plasticizer for polystyrene, the spherical nanoparticles transformed into nanoworms upon cooling to room temperature. The synthesis process is denoted as the temperature directed morphology transformation (TDMT) method, and can be used to produce a wide range of polymer nanoparticles including worms, rods, vesicles, toroids, tadpoles, stacked toroidal nanorattles, other morphologies, or combinations thereof.

The polymer nanoworms were then coupled to the functional groups (O, G, S and C), dialyzed, freeze-dried and then rehydrated with water to make a 1.5 wt % polymer/water dispersion. The polymer (NW_{S,O,C,G}) dispersion was then coated onto surfaces ranging from 1 to 5 sprays. The amount of polymer per area was determined by measuring the dry weight of polymer on the surface of a glass slide using a microbalance.

### Synthesis of the Nanorod

Nanorods can be obtained by temperature directed morphology transformation (TDTM) and ultrasound cutting of the nanoworms. In at least one aspect, a 6 mL latex solution of a nanoworm can be transferred to 2 hot vials (3 mL each) with 60 SL of toluene in each vials. These vials can then be sealed and shaken. The suspensions in these vials can be cooled to 23° C. The solutions can be cooled from 70° C. to 15° C. for about 30 min. The nanostructure can be characterized by transmission electron microscopy (TEM) to confirm the formation of worm-like nanostructures. To form the rods, the worms can be diluted by adding 10 mL of Milli-Q water, and cut using an ultrasound probe (with the pulse of 3 s on and 2 s off as one pulse cycle) for 3 min in an ice-bath at 35% amplitude (3 mm Tapered Micro Tip, VC-750 system from Sonics & Materials). After ultrasound cutting, the nanostructure can be characterized by TEM again to confirm the formation of rods.

Ultrasonic cutting of nanoworms to nanorods can also be carried out by applying probed ultrasound with different pulse cycles (15 seconds on and 10 seconds off as one pulse cycle), (B) 12 cycles (3 min), (C) 36 cycles (9 min) and (D) 48 cycles (12 min).

In at least one aspect, heating a nanoworm or nanorod composition of the present disclosure above the lower critical solution temperature (LCST) (about 37° C.) of the PNIPAM block can produce a gel that when cooled can dissociate back to a sol; a process that is reversible. Nanoworms can form gels at a minimum weight fraction of about 0.1 wt % to about 10 wt %, such as about 1 wt % to about 8 wt %, in an aqueous solution. Nanorods can form gels at a minimum weight fraction of from about 2 wt % to about 16 wt % in an aqueous solution. There is a distribution of lengths (aspect ratios) of nanostructures in a nanostructure sample, and gel formation can depend on the aspect ratio(s) present the nanostructure sample. Without being bound by theory, gels are advantageous because they can be dissociated with increased temperature (such as from room temperature to body temperature of a subject, such as a human) to allow the worm 3-dimensional structure to dissociate and move through the blood.

The weight percentages of the nanorods in water at which the gel can be formed at 37° C. can be measured as follows: generally, the freeze-dried nanorods (e.g., 20 mg) can be redispersed in Milli-Q water by vortexing at 30 wt % in a 1.5 ml Eppendorf tube at 25° C. The tube can then be capped and immersed in a water bath at 37° C. for 2 min. The tube can then be flipped under the water bath to observe the gel formation. Gel formation is defined as no observable flowing of the fluid within 30 seconds. The weight percentage can be lowered by adding more Milli-Q water and vortexing. The gel formation can then be checked again. The minimum weight percentage of the nanorods, for example, in water to form the gel at 37° C. is defined as wt % to form the gel.

### Methods for Depositing Compounds and Compositions

Compounds and compositions of the present disclosure may be deposited onto a surface of an object by any suitable deposition method. A surface of an object may be any suitable surface of any suitable object. A surface may be porous or nonporous. Deposition methods can include one or more of painting, dipping, spraying, marking, taping, brush coating, spin coating, roll coating, doctor-blade coating. Before deposition, a compound or composition of the present disclosure can be diluted in a solvent, such as water. After deposition, the solvent may then evaporate at room temperature forming a compound/composition layer on the object.

In at least one aspect, the object is an interior surface of an aircraft/spacecraft/boat or an air filter surface of an aircraft/spacecraft/boat, such as a surface of an air-conditioning or filtration system. The object can be a floor surface, seat surface, overhead bin surface, ceiling surface, door surface and/or door handle surface of the interior of an aircraft.

In at least one aspect, a compound or composition of the present disclosure is applied, (e.g., sprayed, deposited, printed, etc.) onto a surface of an object for about 1 second to about 10 minutes, such as about 30 seconds to about 2 minutes. In at least one aspect, a compound or composition is applied (e.g., sprayed) onto a surface of an object in an amount of about 1 mL to about 25 kL, such as about 100 L to about 1 kL.

Compounds or compositions of the present disclosure disposed on an object prevents, reduces, and/or eliminates the presence of bacteria and viruses (such as SARS-CoV-2), which can prevent, reduce, and/or eliminate human contact with such bacteria and viruses.

Compositions can have any suitable pH, such as a pH of about 6.5 to about 7.4. For example, a pH of about 6.5 mimics the pH of a mucosal droplet.

Compositions comprising nanostructures (e.g., nanorods or nanoworms) of the present disclosure are advantageous to deposit onto a surface because, for example, an antibacterial and antiviral compound can be applied as a single layer, maintaining efficacy of both compounds. Applying a composition having a nanostructure as a single layer also reduces cost and time of applying the compounds to a surface, as compared to application of two or more layers. By using a water-based solution, reduction of the safety requirements for the end-user and thus time savings and cost savings for application to a surface will be realized. Alternatively, in some examples, thicker layers and/or multiple layers may be applied. In some examples, a surface is refreshed or replenished with one or more additional layers of nanostructure composition at a time after application of a first application (one layer or multiple layers).

In some aspects, methods of disposing a nanostructure onto a surface are described herein. In some aspects, a method includes disposing a layer of a solution or emulsion comprising the nanostructure on the surface. The surface includes a SARS-CoV-2 virus (e.g., BA.1) disposed thereon. The nanostructure includes a compound or salt thereof. The compound includes one or more styrene units, one or more N-alkylacrylamide units, a moiety represented by the formula:

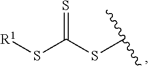

where R¹ is alkyl, a plurality of N,N-(dialkylamino)(divalent alkyl) alkylacrylate units, and a moiety represented by the formula:

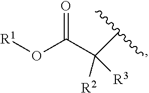

where R¹ is alkyl (branched or linear, substituted or unsubstituted) and R² and R³ are independently hydrogen or alkyl (branched or linear, substituted or unsubstituted).

One or more of the N,N-(dialkylamino)(divalent alkyl)alkylacrylate units has an unsubstituted nitrogen. One or more of the N,N-(dialkylamino)(divalent alkyl)alkylacrylate units is substituted with an octane moiety. One or more of the N,N-(dialkylamino)(divalent alkyl) alkylacrylate units is substituted with a moiety selected from the group consisting of guanidine, polygalactose, coumarin, and combination(s) thereof. In some examples, a BA.1 SARS-CoV-2 virus is B.1.1.529 SARS-CoV-2 virus. The surface to be treated with the coating can be any suitable surface of any suitable object. In some non-limiting aspects, an object is a mask and a surface is an interior portion of a fuselage of an aircraft, or any other suitable surface. In some examples, a surface is a surface (interior or exterior) of an aircraft, a ship, a train, a terminal (e.g., bus, train, airport, etc.), or a spacecraft.

The emulsion or solution has a concentration of the nanostructure of about 0.5 wt % to about 3 wt %.

### Methods for Use as a Pharmaceutical Drug

In some aspects, the present disclosure further provides methods for treating a condition in a subject having or susceptible to having such a condition, by administering to the subject a therapeutically-effective amount of one or more compounds or compositions of the present disclosure. In one aspect, the treatment is preventative treatment. In another aspect, the treatment is palliative treatment. In another aspect, the treatment is restorative treatment.

A method for treating a condition can include administering to a subject a therapeutically effective amount of a nanostructure, or pharmaceutically acceptable salt thereof (or a composition having a nanostructure, or pharmaceutically acceptable salt thereof).

Methods for treating a condition are described herein. In some aspects, a method includes administering to a subject a therapeutically effective amount of a nanostructure. The nanostructure includes a compound, or a pharmaceutically acceptable salt thereof. The condition includes a viral infection as a result of a BA.1 SARS-CoV-2 virus. The compound includes one or more styrene units, one or more N-alkylacrylamide units, a moiety represented by the formula:

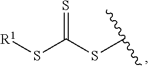

where R¹ is alkyl, a moiety represented by the formula

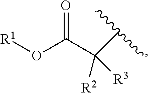

where R¹ is alkyl (branched or linear, substituted or unsubstituted) and R² and R³ are independently hydrogen or alkyl (branched or linear, substituted or unsubstituted), and a plurality of N,N-(dialkylamino)(divalent alkyl)alkylacrylate units, where one or more of the N,N-(dialkylamino)(divalent alkyl)alkylacrylate units has an unsubstituted nitrogen. One or more of the N,N-(dialkylamino)(divalent alkyl)alkylacrylate units is substituted with an octane moiety. One or more of the N,N-(dialkylamino)(divalent alkyl)alkylacrylate units is substituted with a moiety selected from the group consisting of guanidine, polygalactose, coumarin, and combination(s) thereof.

A coating described herein can be scaled and applied directly to surfaces as a water solution to act as an effective virucidal agent that renders SARS-CoV-2 variants of concern non-infectious. The design of the polygalactose (e.g., about 2 to about 20 galactose units) attached to the polymer nanostructure and potential specific bonding interactions with highly glycosylated SARS-CoV-2 provide a binding motif independent of the virus variant and mutations found in the virus Spike attachment glycoprotein. In some aspects, a polygalactose has greater than 20 galactose units, e.g., up to about 1,000 galactose units. The polygalactose binding in combination with the octane moieties and the responsive nature of the nanostructures that mechanically attach to and disrupt the viral particles, rendering them non-infectious. It was further shown that the SARS-CoV-2 viral RNA genome may either degrade as a result of the guanadine groups or be electrostatically captured by the cationic groups attached to the polymer that then allows natural degradation. The fact that the viral RNA genome cannot be detected after interaction of the viruses with the polymer coated surfaces demonstrates complete virucidal activity of the polymer. It is believed that the polymer coating will provide inactivation of newly emerging SARS-CoV-2 variants of concern while still maintaining the ability to be re-designed to target other viruses. Finally, the polymer was found to be non-toxic by oral ingestion in rats and had little or no skin sensitization when applied on the skin of mice, indicating the potential safe use as a component of personal protective equipment or high touch-point surfaces that comes into contact with skin. The nanostructure composition can also be administered to subjects as a therapeutic treatment.

### 1. Conditions

The conditions that can be treated in accordance with the present disclosure include, but are not limited to, conditions caused by a toxin (such as an antigen) and inflammatory disorders such as septic shock. The conditions that can be treated in accordance with the present disclosure include, but are not limited to viral infections, bacterial infections, chronic inflammatory disorders, acute inflammatory disorders, and cancers. In some aspects, the condition to be treated includes a bacterial infection, a viral infection, or a cancer immunotherapy. Cancer immunotherapy can include cervical cancers such as those resulting from an infection of the cervix with human papillomavirus.

Viral infections can include those caused by Ebola, influenza, SARS (such as SARS CoV-2), Noro (gastro), or Zika. Viral infections can include viral respiratory infections (e.g., of the nose, throat, upper airways, or lungs) such as pneumonia, laryngotracheobronchitis, bronchiolitis. Viral infections can include viral gastrointestinal infections such as gastroenteritis caused by a norovirus or rotavirus. Viral infections can include viral liver infections such as hepatitis. Viral infections can include viral nervous system infections such as encephalitis caused by rabies or West Nile virus. Viral infections include warts and/or infections caused by human papilloma virus (HPV). Viral infections can include infections that cause cancer such as infections caused by Epstein-Barr virus, Hepatitis B, Hepatitis C, Herpesvirus 8, or Human papillomavirus. Symptoms of viral infections can include fever, muscle aches, coughing, sneezing, runny nose, headache, chills, diarrhea, vomiting, rash, or weakness.

Bacterial infections can include pneumonia, meningitis, food poisoning, and bacterial skin infections such as those caused by Staphylococcus or Streptococcus, cellulitis, folliculitis, impetigo, and boils. Bacterial infections (e.g., by food poisoning) can include infections caused by Escherichia coli (E. coli), Campylobacter jejuni (C. jejuni), Clostridium botulinum (C. botulinum), Listeria monocytogenes (L. monocytogenes), Salmonella, and Vibrio. Bacterial infections can include bacterial meningitis, otitis media, urinary tract infection, and respiratory tract infections such as sore throat, bronchitis, sinusitis, and pneumonia. Symptoms of bacterial infections can include nausea, vomiting, diarrhea, fever, chills, and abdominal pain.

In some aspects, the methods described herein are used to treat patients with disorders arising from dysregulated cytokine, enzymes and/or inflammatory mediator production, stability, secretion, posttranslational processing. Examples of cytokines that may be dysregulated include interleukins 1, 2, 6, 8, 10, 12, 17, 22, and 23 along with tumor necrosis factor alpha and interferons alpha, beta, and gamma. Examples of inflammatory mediators that may be dysregulated include nitric oxide, prostaglandins, and leukotrienes. Examples of enzymes include cyclo-oxygenase, nitric oxide synthase, and matrixmetalloprotease.

Examples of inflammatory conditions relevant to the technology include, but are not limited to, sepsis, septic shock, endotoxic shock, exotoxin-induced toxic shock, gram negative sepsis, and toxic shock syndrome. Inflammatory conditions can include those experienced by immunosuppressed individuals, and can also include “superbugs”, including bacterial and viral strains resistant to current therapeutics.

### 2. Subjects

Suitable subjects to be treated according to the present disclosure include mammalian subjects. Mammals according to the present disclosure include, but are not limited to, human, canine, feline, bovine, caprine, equine, ovine, porcine, rodents, lagomorphs, primates, and the like, and encompass mammals in utero. Subjects may be of either gender and at any stage of development.

### 3. Administration and Dosing

Compounds or compositions of the present disclosure can be administered to a subject in a therapeutically effective amount.

Compounds or compositions of the present disclosure can be administered by any suitable route in the form of a pharmaceutical composition adapted to such a route, and in a dose effective for the treatment intended. An effective dosage is typically in the range of about 0.001 to about 100 mg per kg body weight per day, such as about 0.01 to about 30 mg/kg/day, in single or divided doses. Depending on age, species and condition being treated, dosage levels below the lower limit of this range can be suitable. In other cases, still larger doses can be used without side effects. Larger doses can also be divided into several smaller doses, for administration throughout the day.

### Pharmaceutical Compositions

For the treatment of the conditions referred to above, the compounds described herein can be administered as follows:

### Oral Administration

Compounds or compositions of the present disclosure can be administered orally, including by swallowing, so that the compound enters the gastrointestinal tract, or absorbed into the blood stream directly from the mouth (e.g., buccal or sublingual administration).

Suitable compositions for oral administration include solid formulations such as tablets, lozenges and capsules, which can contain liquids, gels, or powders. Compositions for oral administration may be formulated as immediate or modified release, including delayed or sustained release, optionally with enteric coating.

Liquid formulations can include solutions, syrups and suspensions, which can be used in soft or hard capsules. Such formulations can include a pharmaceutically acceptable carrier, for example, water, ethanol, polyethylene glycol, cellulose, or an oil. The formulation can also include one or more emulsifying agents and/or suspending agents.

In a tablet dosage form the amount of a compound present can be from about 0.05% to about 95% by weight, such as about 2% to about 50% by weight of the dosage form. In addition, tablets may contain a disintegrant, comprising about 0.5% to about 35% by weight, such as about 2% to about 25% of the dosage form. Examples of disintegrants include: methyl cellulose, sodium or calcium carboxymethyl cellulose, croscarmellose sodium, polyvinylpyrrolidone, hydroxypropyl cellulose, or starch.

Suitable lubricants, for use in a tablet, can be present in amounts of about 0.1% to about 5% by weight. Lubricants can include calcium, zinc or magnesium stearate, or sodium stearyl fumarate.

Suitable binders, for use in a tablet, include gelatin, polyethylene glycol, sugars, gums, starch, hydroxypropyl cellulose and the like. Suitable diluents, for use in a tablet, include mannitol, xylitol, lactose, dextrose, sucrose, sorbitol, or starch.

Suitable surface active agents and glidants, for use in a tablet, may be present in amounts from about 0.1% to about 3% by weight. Surface active agents and glidants can include polysorbate 80, sodium dodecyl sulfate, talc, or silicon dioxide.

### Parenteral Administration

Compounds and compositions of the present disclosure can be administered directly into the blood stream, muscle, or internal organs. Suitable methods for parenteral administration can include intravenous, intra-muscular, subcutaneous intraarterial, intraperitoneal, intrathecal, or intracranial. Suitable devices for parenteral administration include injectors (including needle and needle-free injectors) and infusion methods.

Compositions for parenteral administration can be formulated as immediate or modified release, including delayed or sustained release.

Most parenteral formulations are aqueous solutions containing excipients, including salts, buffering agents and carbohydrates.

Parenteral formulations can also be prepared in a dehydrated form (e.g., by lyophilization) or as sterile non-aqueous solutions. These formulations can include water. Solubility-enhancing agents can also be used in preparation of parenteral solutions.

### Topical Administration

Compounds and compositions of the present disclosure can be administered topically to the skin or transdermally. Formulations for this topical administration can include lotions, solutions, creams, gels, hydrogels, ointments, foams, implants, patches and the like. Pharmaceutically acceptable carriers for topical administration formulations can include water, alcohol, mineral oil, glycerin, polyethylene glycol and the like. Topical administration can be performed by electroporation, iontophoresis, or phonophoresis.

Compositions for topical administration can be formulated as immediate or modified release, including delayed or sustained release.

### Combinations and Combination Therapy

The compounds and compositions of the present disclosure can be used, alone or in combination with other pharmaceutically active compounds, to treat conditions such as those previously described above. The compound(s)/composition(s) of the present disclosure and other pharmaceutically active compound(s) can be administered simultaneously (either in the same dosage form or in separate dosage forms) or sequentially. Accordingly, in one aspect, the present disclosure includes methods for treating a condition by administering to the subject a therapeutically-effective amount of one or more compounds of the present disclosure and one or more additional, different pharmaceutically active compounds.

In another aspect, there is provided a pharmaceutical composition comprising one or more compounds of the present disclosure, one or more additional pharmaceutically active compounds, and a pharmaceutically acceptable carrier.

In another aspect, the one or more additional, different pharmaceutically active compounds is one or more anti-inflammatory drugs, anti-atherosclerotic drugs, immunosuppressive drugs, immunomodulatory drugs, cytostatic drugs, anti-proliferative agents, angiogenesis inhibitors, kinase inhibitors, cytokine blockers, or inhibitors of cell adhesion molecules.

Compounds and compositions of the present disclosure can also be used in combination with other therapeutic reagents that are selected for their therapeutic value for the condition to be treated. In general, the compounds and compositions described herein and, in aspects where combinational therapy is employed, other agents do not have to be administered in the same pharmaceutical composition, and, because of different physical and chemical characteristics, are optionally administered by different routes. The initial administration is generally made according to established protocols, and then, based upon the observed effects, the dosage, modes of administration and times of administration subsequently modified. In certain instances, it is appropriate to administer a compound of the present disclosure as described herein in combination with another, different therapeutic agent. By way of example only, if one of the side effects experienced by a patient upon receiving a compound of the present disclosure is rash, then it is appropriate to administer an anti-histamine agent in combination with the initial therapeutic agent. Or, by way of example only, the therapeutic effectiveness of a compound of the present disclosure is enhanced by administration of another therapeutic agent (which also includes a therapeutic regimen) that also has therapeutic benefit. Regardless of the disease, disorder or condition being treated, the overall benefit experienced by the patient is either simply additive of the two therapeutic agents or the patient experiences a synergistic benefit.

Therapeutically effective dosages vary when the drugs are used in treatment combinations. Methods for experimentally determining therapeutically effective dosages of drugs and other agents for use in combination treatment regimens are documented methodologies. Combination treatment further includes periodic treatments that start and stop at various times to assist with the clinical management of the patient. In any case, the multiple therapeutic agents (one of which is a compound of the present disclosure) are administered in any order, or even simultaneously. If simultaneously, the multiple therapeutic agents are optionally provided in a single, unified form, or in multiple forms (by way of example only, either as a single pill or as two separate pills).

In some aspects, one of the therapeutic agents is given in multiple doses, or both are given as multiple doses. If not simultaneous, the timing between the multiple doses optionally varies from more than zero weeks to less than twelve weeks.

In addition, the combination methods, compositions and formulations are not to be limited to the use of only two agents, the use of multiple therapeutic combinations are also envisioned. It is understood that the dosage regimen to treat, prevent, or ameliorate the condition(s) for which relief is sought, is optionally modified in accordance with a variety of factors. These factors include the disorder from which the subject suffers, as well as the age, weight, sex, diet, and medical condition of the subject. Thus, the dosage regimen actually used can vary widely, in some aspects, and therefore can deviate from the dosage regimens set forth herein.

The pharmaceutical agents which make up the combination therapy disclosed herein are optionally a combined dosage form or in separate dosage forms intended for substantially simultaneous administration. The pharmaceutical agents that make up the combination therapy are optionally also administered sequentially, with either agent being administered by a regimen calling for two-step administration. The two-step administration regimen optionally calls for sequential administration of the active agents or spaced-apart administration of the separate active agents. The time period between the multiple administration steps ranges from, a few minutes to several hours, depending upon the properties of each pharmaceutical agent, such as potency, solubility, bioavailability, plasma half-life and kinetic profile of the pharmaceutical agent. Circadian variation of the target molecule concentration is optionally used to determine the optimal dose interval.

Compounds of the present disclosure or compositions having a compound of the present disclosure can be used (e.g., administered) in combination with drugs from the following classes: NSAIDs, immunosuppressive drugs, immunomodulatory drugs, cytostatic drugs, anti-proliferative agents, angiogenesis inhibitors, biological agents, steroids, vitamin D3 analogs, retinoids, other kinase inhibitors, cytokine blockers, corticosteroids and inhibitors of cell adhesion molecules. Where a subject is suffering from or at risk of suffering from atherosclerosis or a condition that is associated with atherosclerosis, a compound or composition of the present disclosure can be optionally used together with one or more agents or methods for treating atherosclerosis or a condition that is associated with atherosclerosis in any combination. Examples of therapeutic agents/treatments for treating atherosclerosis or a condition that is associated with atherosclerosis include, but are not limited to any of the following: torcetrapib, aspirin, niacin, HMG CoA reductase inhibitors (e.g., atorvastatin, fluvastatin, lovastatin, pravastatin, rosuvastatin and simvastatin), colesevelam, cholestyramine, colestipol, gemfibrozil, probucol and clofibrate.

Where a subject is suffering from or at risk of suffering from an inflammatory condition, a compound or composition of the present disclosure is optionally used together with one or more agents or methods for treating an inflammatory condition in any combination. Examples of therapeutic agents/treatments for treating an autoimmune and/or inflammatory condition include, but are not limited to any of the following: corticosteroids, nonsteroidal antiinflammatory drugs (NSAID) (e.g. ibuprofen, naproxen, acetaminophen, aspirin, Fenoprofen (NALFON®), Flurbiprofen (ANSAID®), Ketoprofen, Oxaprozin (DAYPRO®), Diclofenac sodium (VOLTAREN®), Diclofenac potassium (CATAFLAM®), Etodolac (LODINE®), Indomethacin (INDOCIN®), Ketorolac (TORADOL®), Sulindac (CLINORIL®), Tolmetin (TOLECTIN®), Meclofenamate (MECLOMEN®), Mefenamic acid (PONSTEL®), Nabumetone (RELAFEN®), Piroxicam (FELDENE®), cox-2 inhibitors (e.g., celecoxib (CELEBREX®))), immunosuppressants (e.g., methotrexate (RHEUMATREX®), leflunomide (ARAVA®), azathioprine (IMURAN®), cyclosporine (NEORAL®, SANDINMUNE®), tacrolimus and cyclophosphamide (CYTOXAN®), CD20 blockers (RITUXIMAB®), Tumor Necrosis Factor (TNF) blockers (e.g., etanercept (ENBREL®), infliximab (REMICADE®) and adalimumab (HUMIRA®)), Abatacept (CTLA4-Ig) and interleukin-1 receptor antagonists (e.g. Anakinra (KINERET®), interleukin 6 inhibitors (e.g., ACTEMRA®), interleukin 17 inhibitors (e.g., AIN457), Janus kinase inhibitors (e.g., Tasocitinib), syk inhibitors (e.g. R788), chloroquine and its derivatives.

For use in cancer and neoplastic diseases a compound or composition of the present disclosure is optionally used together with one or more of the following classes of drugs: wherein the anti-cancer agent is an EGFR kinase inhibitor, MEK inhibitor, VEGFR inhibitor, anti-VEGFR2 antibody, KDR antibody, AKT inhibitor, PDK-1 inhibitor, PI3K inhibitor, c-kit/Kdr tyrosine kinase inhibitor, Bcr-Abl tyrosine kinase inhibitor, VEGFR2 inhibitor, PDGFR-beta inhibitor, KIT inhibitor, Flt3 tyrosine kinase inhibitor, PDGF receptor family inhibitor, Flt3 tyrosine kinase inhibitor, RET tyrosine kinase receptor family inhibitor, VEGF-3 receptor antagonist, Raf protein kinase family inhibitor, angiogenesis inhibitor, Erb2 inhibitor, mTOR inhibitor, IGF-1R antibody, NFkB inhibitor, proteosome inhibitor, chemotherapy agent, or glucose reduction agent.

### EXAMPLES

Reagents: Unless otherwise stated, all chemicals were used as received. The solvents used were of either HPLC or AR grade; these included dichloromethane (DCM, Aldrich AR grade), DMSO (Aldrich, 99.9%), n-hexane (Emsure, ACS), chloroform (Emsure, ACS), methanol (Merck, Emsure, ACS), acetonitrile (LiChrosolv, hypergrade for LC-MS), petroleum spirit (BR 40-60° C., Univar, AR), toluene (Merck, for analysis EMSURE ACS, ISO, Reag. Ph Eur), ethyl acetate (ChemSupply, AR), ethanol (ChemSupply, AR), N,N-dimethylformamide (DMF: Labscan, AR grade), and N,N-dimethylacetamide (Aldrich, >99%). Activated basic alumina (Aldrich: Brockmann I, standard grade, 150 mesh, 58 Å), silica gel (Aldrich, 230-400 mesh, 60 Å), magnesium sulphate (anhydrous, Aldrich), Milli-Q water (Biolab, 18.2 MQm), sodium dodecyl sulphate (SDS, Aldrich, 99%), 1-butanethiol (Aldrich, 99%), D-(+)-galactose (Aldrich, ≥99%), propargyl bromide solution (Aldrich, 80 wt. % in toluene, contains 0.3% magnesium oxide as stabilizer), lithium chloride (Aldrich, 99%), tripotassium phosphate (Aldrich, ≥98%), potassium hydroxide (Aldrich), 3-chloropropylamine hydrochloride (Aldrich, 98%), triethylamine (Aldrich, ≥99.5%), acryloyl chloride (Merck, stabilized with phenothiazine), sodium hydrogen carbonate (Aldrich, 99.5%), sodium azide (Aldrich, ≥99.5%), hydrochloric acid (36%, Ajax, AR), sulfuric acid (Aldrich, 98%), trifluoroacetic acid (Merck, >99%), carbon disulfide (Aldrich, >99.9%), methyl-2-bromopropionate (MBP, Aldrich, 98%), 2-ethyl-2-thiopseudourea hydrobromide (Aldrich, 98%), iodooctane (Aldrich, 98%), copper (II) sulfate (Aldrich, 99%), copper (II) sulfate anhydrous powder (Aldrich, ≥99.99% trace metals basis), Cu(0) powder (Aldrich, <425 m, 99.5% trace metals basis), and L-ascorbic acid (Aldrich, 99%) were used as received.

Monomers, initiator, and ligand: N-isopropylacrylamide (NIPAM, Aldrich, 97%) and N,N-(dimethylamino)ethyl methacrylate (DMAEMA, Aldrich, 98%) were dissolved in ethanol with activated basic alumina and after filtration used directly for the synthesis of macro chain transfer agents (MacroCTAs). Styrene (STY, Aldrich, >99%) was passed through a basic alumina column to remove inhibitor. Azobisisobutyronitrile (AIBN, Riedel-de Haen) was recrystallized from methanol twice prior to use. Tris(2-(dimethylamino)ethyl)amine (Me₆TREN),₁ Cu(II)Br₂/Me₆TREN complex,₂ 3-azido-7-hydroxycoumarin azide (coumarin azide)₃ were synthesized according to literature procedures.

RAFT agent: Methyl 2-(butylthiocarbonothioylthio) propanoate (MCEBTTC) RAFT agent was synthesized according to the literature procedure.

Nuclear Magnetic Resonance (NMR) All NMR spectra were recorded on either Bruker DRX 400 or 500 MHz spectrometers using an external lock (CDCl₃, DMSO0-d6 or D₂O).

Size Exclusion Chromatography (SEC) and Triple Detection-Size Exclusion Chromatography (TD-SEC): Analysis of the molecular weight distributions of the polymers was determined using a Polymer Laboratories GPC50 Plus equipped with differential refractive index detector. Absolute molecular weights of polymers were determined using a Polymer Laboratories GPC50 Plus equipped with dual angle laser light scattering detector, viscometer, and differential refractive index detector. HPLC grade N,N-dimethylacetamide (DMAc, containing 0.03 wt % LiCl) was used as the eluent at a flow rate of 1.0 mL/min. Separations were achieved using two PLGel Mixed B (7.8×300 mm) SEC columns connected in series and held at a constant temperature of 50° C. InfinityLab EasiVial polystyrene standards were used for SEC column calibration. Samples of known concentration were freshly prepared in DMAc+0.03 wt % LiCl and passed through a 0.45 m PTFE syringe filter prior to injection. The absolute molecular weights and dn/dc values were determined using Polymer Laboratories Multi Cirrus software based on the quantitative mass recovery technique.

Dynamic Light Scattering (DLS): The size and zeta potential of particles was measured by DLS which was performed using a Malvern Zetasizer Nano Series running DTS software and operating a 4 mW He—Ne laser at 633 nm. Analysis was performed at an angle of 1730 and a constant temperature of 25° C. The number-average hydrodynamic particle size and PDI(DLs) are reported. The PDI_{(DLS)} was used to describe the width of the particle size distribution, and calculated from a Cumulants analysis of the DLS measured intensity autocorrelation function and is related to the standard deviation of the hypothetical Gaussian distribution (i.e., PDI_{(DLS)}=σ₂/Z_{D2}, where σ is the standard deviation and Z_{D} is the Z average mean size).

Transmission Electron Microscopy (TEM) The nanostructure appearance was determined using a HT-7700 transmission electron microscope utilizing an accelerating voltage of 80 kV with spot size 1 at ambient temperature. A typical TEM grid preparation was as follows: A sample was diluted with Milli-Q water to approximately 0.02-0.05. wt % at room temperature. A formvar precoated copper TEM grid was dipped into the solution, the excess aliquot was blotted and then allowed to air dry prior imaging on TEM.

Attenuated Total Reflectance-Fourier Transform Spectroscopy (ATR-FTIR) ATR-FTIR spectra were obtained using a horizontal, single bounce, diamond ATR accessory on a Nicolet Nexus 870 FT-IR. Spectra were recorded between 4000 and 500 cm₋₁ for 32 scans at 4 cm₋₁ resolution with an OPD velocity of 0.6289 cm s₋₁. Solids were pressed directly onto the diamond internal reflection element of the ATR without further sample preparation.

### Synthesis of Guanidine Azide—Synthesis of 3-Azidopropylamine

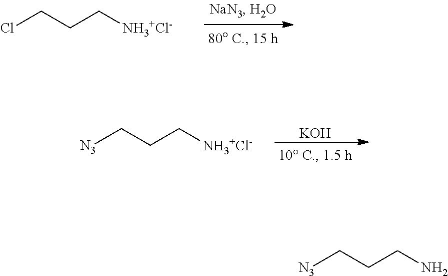

Sodium azide (15 g, 2.31×10₋₁ mol) was dissolved in Milli-Q water (80 mL) in the 250 mL round bottom flask. 3-chloropropylamine hydrochloride (10 g, 7.69×10₋₂ mol) was added to the reaction solution. The reaction was refluxed at 80° C. for 15 h, then gradually cooled to ˜10° C., KOH (6.15 g, 1.1×10₋₁ mol) added and the reaction mixture stirred for 1.5 h. The reaction mixture was then placed in the separatory funnel and the product was extracted with diethyl ether (5×100 mL). The organic phase was dried using MgSO₄, and diethyl ether was removed using rotary evaporation to isolate the product as a light brown liquid. Yield=87%. ¹H NMR (500 MHz, CDCl₃, 298K) δ (ppm): 3.37 (t, ₃J=6.5 Hz, 2H, H₂NCH₂CH₂), 2.81 (t, ₃J=6.75 Hz, 2H, CH₂CH₂N₃), 1.73 (quint, ₃J=6.9 Hz, 2H, H₂NCH₂CH₂). ₁₃C NMR (125 MHz, CDCl₃, 298K) δ (ppm): 49.3 (CH₂CH₂N₃), 39.4 (H₂NCH₂CH₂), 32.4 (H₂NCH₂CH₂).

### Synthesis of Guanidine Azide

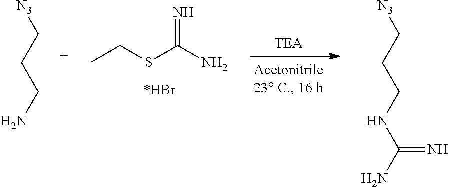

Azidopropylamine (3.11 g, 3.11×10₋₂ mol) was added dropwise to a 50-mL round-bottomed flask charged with 2-ethyl-2-thiopseudourea hydrobromide (5 g, 2.70×10₋₂ mol), triethylamine (3.77 mL, 2.73 g, 2.70×10⁻² mol), 24 mL of acetonitrile, and a teflon-coated stir bar. 1.2 mL of DI H₂O was added to the solution to fully dissolve the mixture. The reaction was stirred at 23° C. for 16 h. The reaction was stopped, and the solvent was removed by rotary evaporation to produce a viscous clear oil. The product was purified via column chromatography using 50:50 EtOH/EtOAc as the mobile phase over silica, R_{f}=0.59. Yield=90%. ₁H NMR (400 MHz, D₂O, 298K) δ (ppm): 3.46 (t, ₃J=6.6 Hz, 2H, CH₂CH₂NH), 3.31 (t, ₃J=6.8 Hz, 2H, N₃CH₂CH₂), 1.87 (quint, ₃J=6.7 Hz, 2H, N₃CH₂CH₂). ₁₃C NMR (100 MHz, DMSO-d6, 298K) δ (ppm): 156.9 (NHC(NH)NH₂), 48.0 (N₃CH₂CH₂), 38.2 (CH₂CH₂NH), 27.7 (N₃CH₂CH₂).

### Synthesis of polygalactose azide—Synthesis of 1,2:3,4-Di-O-isopropylidene-D-galactopyranose

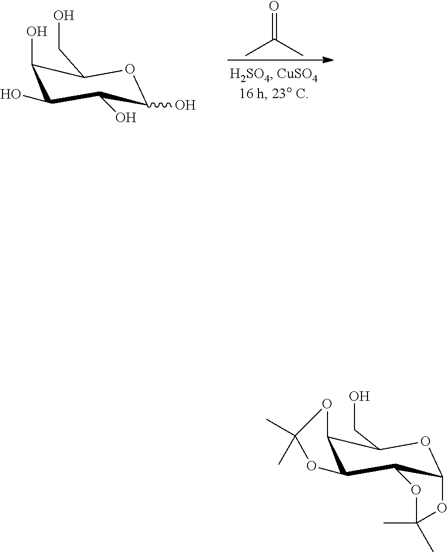

Anhydrous CuSO₄ (40 g, 0.251 mol) was dissolved in acetone (360 mL) and stirred under argon. Concentrated H₂SO₄ (2 mL) and D-galactose (27.36 g, 0.152 mol) were added to the solution in sequence and stirred at room temperature overnight. A suspension of NaHCO₃ (54 g, 0.643 mol) in water (100 mL) was added to the mixture. The precipitate was removed by filtration and washed with acetone. The washings and filtrates were combined, and the solution was subjected to rotary evaporation to remove acetone. The product was extracted with diethyl ether (5×100 mL) from the aqueous phase. The combined ether solution was dried over anhydrous magnesium sulfate and then rotary evaporated. The product was a clear viscous oil. Yield=60%. 1H NMR (400 MHz, CDCl₃, 298K) δ (ppm): 5.57 (d, 3J=4.8 Hz, 1H, H-1), 4.61 (dd, 3J=7.94 Hz, 3J=2.34 Hz, 1H, H-3), 4.34 (dd, 3J=5.0 Hz, 3J=2.36 Hz, 1H, H-2), 4.27 (dd, 3J=7.9 Hz, 3J=0.98 Hz, 1H, H-4), 3.90-3.83 (m, 2H, H₂-6), 3.78-3.72 (m, 1H, H-5), 2.17-2.14 (m, 1H, OH), 1.53 (s, 3H, CH₃), 1.46 (s, 3H, CH₃), 1.34 (s, 6H, 2×CH₃). 13C NMR (100 MHz, CDCl₃, 298K) δ (ppm): 109.6 (C(CH₃)₂O₂), 108.8 (C(CH₃)₂O₂), 96.4 (C-1), 71.7 (C-4), 70.9 (C-3), 70.7 (C-2), 68.3 (C-5), 62.4 (C-6), 26.14 (CH₃), 26.05 (CH₃), 25.1 (CH₃), 24.4 (CH₃).

### Synthesis of 6-O-acryloyl-1,2:3,4-di-O-isopropylidene-D-galactopyranose

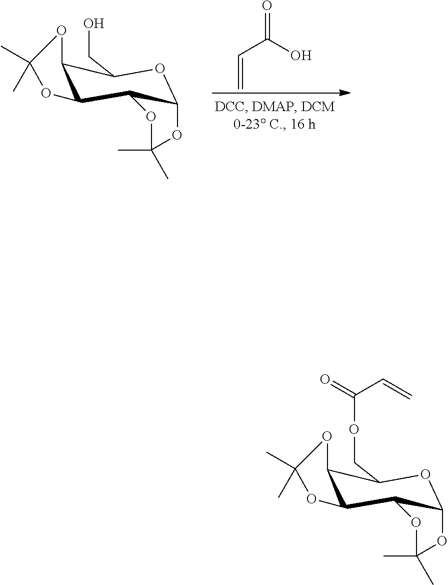

1,2:3,4-Di-O-isopropylidene-D-galactopyranose (19.11 g, 0.073 mol), acrylic acid (6.35 g, 0.088 mol) and DMAP (0.987 g, 0.0081 mol) was dissolved in 150 mL of dry DCM in the round bottom flask. The round bottom flask was placed inside an ice-bath for 20 min. The solution of DCC (18.18 g, 0.088 mol) in dry DCM (80 mL) was added dropwise to the reaction mixture and the reaction was carried out for 16 h. Then, the reaction mixture was filtered to remove DCU and DCM was removed from the filtrate by rotary evaporation. The crude product was purified via column chromatography using 4:1 Petroleum Spirit/Ethyl Acetate as the mobile phase over silica, R_{f}=0.57. The product is white crystal. Yield=33%. ¹H NMR (400 MHz, CDCl₃, 298K) δ (ppm): 6.43 (dd, 2J=1.4 Hz, ₃J=17.3 Hz, 1H, —CH═C_{E}H_{Z}H), 6.16 (dd, ₂J=10.4 Hz, ₃J=17.2 Hz, 1H, —CH═C_{E}H_{Z}H), 5.83 (dd, ₂J=1.2 Hz, ₃J=10.4 Hz, 1H, —CH═C_{E}H_{Z}H), 5.54 (d, ₃J=4.8 Hz, 1H, H-1), 4.63 (dd, ₃J=7.6 Hz, ₃J=2.4 Hz, 1H, H-3), 4.38 (dd, ₃J=11.6 Hz, ₃J=4.76 Hz, 1H, H-6), 4.33 (dd, ₃J=4.96 Hz, ₃J=2.5 Hz, 1H, H-2), 4.29-4.24 (m, 2H, H-6, H-4), 4.08-4.05 (m, 1H, H-5), 1.51 (s, 3H, CH₃), 1.45 (s, 3H, CH₃), 1.34 (s, 3H, CH₃), 1.33 (s, 3H, CH₃). ₁₃C NMR (100 MHz, CDCl₃, 298K) δ (ppm): 166.2 (C(O)CH═CH₂), 131.2 (C(O)CH═CH₂), 128.3 (C(O)CH═CH₂), 109.8 (C(CH₃)₂O₂), 108.9 (C(CH₃)₂O₂), 96.4 (C-1), 71.2 (C-4), 70.8 (C-3), 70.6 (C-2), 66.1 (C-5), 63.6 (C-6), 26.11 (CH₃), 26.08 (CH₃), 25.1 (CH₃), 24.6 (CH₃).

### SET-LRP of 6-O-acryloyl-1,2:3,4-di-O-isopropylidene-D-galactopyranose

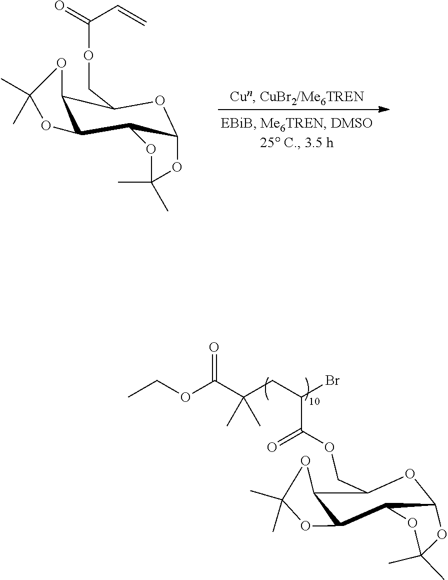

Protected galactose acrylate (7.59 g, 2.42×10₋₂ mol), Me₆tren (65 μL, 2.42×10₋₄ mol), CuBr₂/Me₆tren (0.1096 g, 2.42×10₋₄ mol) and DMSO (25 mL) were added to a 50 mL flask, cooled down in an ice-bath and purged with argon for 60 min to remove oxygen. Cu(0) powder (0.015 g, 2.42×10₋₄ mol) was added to a 100 mL Schlenk flask and purged with argon for 60 min. Then, degassed solution of the initiator EBiB (355 μL, 2.42×10⁻³ mol) in DMSO (5 mL) was added to a 50 mL flask via a degassed syringe. The reaction mixture from a 50 mL flask was transferred to a Schlenk flask via a degassed syringe. A Schlenk tube was placed into a temperature-controlled oil bath at 25° C. The reaction was stopped after 3 h 15 min by quenching in liquid nitrogen. Then, 20 mL of acetone was added to redissolve reaction mixture and this solution was passed through basic alumina column. The column was washed a few times with acetone. Then, acetone was evaporated using a rotavap and the residue was used directly for the azidation step. Small amount of polymer (40 mg) was purified by precipitation in Milli-Q water and subsequent freeze-dry for SEC and ₁H NMR analyses. Conversion (¹H NMR, CDCl₃, 298K)=100%; SEC (RI, DMAc+0.03 wt. % LiCl, PSTY standards): Mₙ=2720, Ð=1.12; ₁H NMR (400 MHz, CDCl₃, 298K): N (repeating units)=10, Mₙ=3340.

### Synthesis of protected polygalactose-N₃

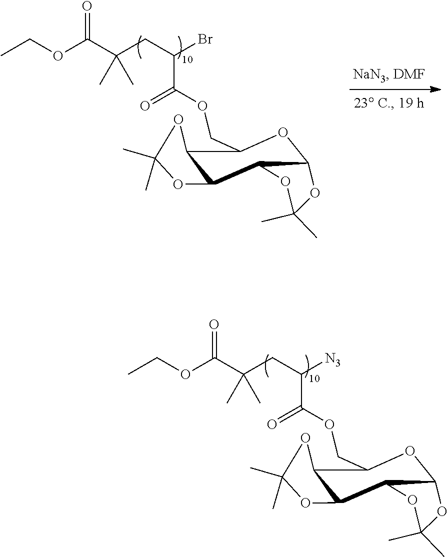

Polygalactose-Br (8.065 g, 2.42×10₋₃ mol) was dissolved in 240 mL of DMF. Then NaN₃ (3.14 g, 4.83×10₋₂ mol) was added to the polymer solution. The reaction was carried out for 19 h. DMF was removed by nitrogen flow overnight. The resulting mixture was redissolved in 200 mL of chloroform and the insoluble salts were filtered out. The chloroform phase was washed with Milli-Q water three times (3×50 mL). The chloroform phase was dried with MgSO₄ and chloroform was removed by rotary evaporation to yield a white powder as a product. SEC (RI, DMAC+0.03 wt. % LiCl, PSTY standards): Mₙ=2620, Ð=1.13.

### Deprotection of Protected polygalactose-N₃

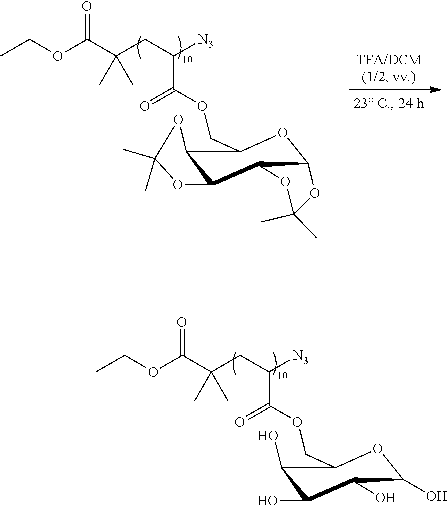

The polymer (8 g, 4.85×10₋₂ mol of acetal groups) was dissolved in 107 mL of DCM. Then, 53.43 mL of TFA (0.698 mol, 14.4 eq.) was added. The reaction was stirred for 24 h (3 mL of Milli-Q water was added 1 h before the reaction was stopped). After that the reaction mixture was concentrated by nitrogen flow and precipitated into diethyl ether. After that, the solid was redissolved in acetone and precipitated again into diethyl ether. The polymer was isolated by centrifugation and dried under high vacuum for 8 h at 25° C. ¹H NMR (400 MHz, D₂O, 298K): % of deprotection=100%.

### Synthesis of MacroCTAs—MacroCTA-A (PNIPAM45-S(C═S)SC4H9)

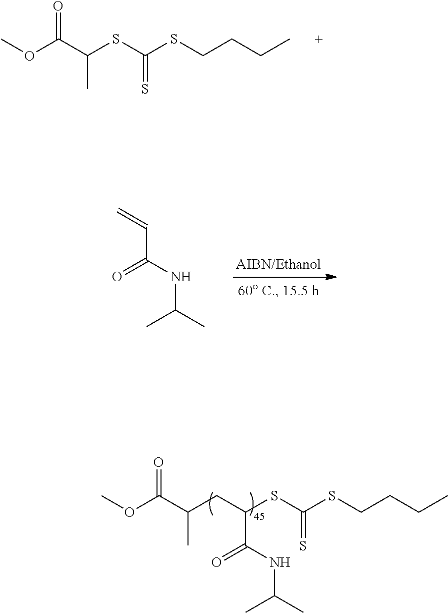

NIPAM (15.1 g, 1.33×10₋₁ mol) was dissolved in 30.3 mL of ethanol and stirred with basic alumina (100 mg) for 30 min to remove inhibitor. The mixture was filtered, and MCEBTTC RAFT agent (0.75 g. 2.97×10₋₃ mol) and AIBN (0.073 g, 4.64×10₋₄ mol) were added. The solution was degassed with argon for 40 min. Polymerization of MacroCTA-A was carried out at 60° C. for 15.5 h. The reaction was quenched by exposure to air and used directly in the emulsion polymerization step. A small portion of the polymer crude was taken for ₁H NMR characterization (to determine NIPAM conversion). Another small portion of the polymer crude was taken and purified via dialysis against Milli-Q water (3500 MWCO), freeze dried, then characterized by ₁H NMR and SEC (to determine repeating units and molecular weight distribution of the polymer).

TABLE S1
Synthesis of MacroCTA-A by RAFT-mediated polymerization.

| | Conv. | Mₙ | | | | Repeating |
|---|---|---|---|---|---|---|
| | (%) | | ¹H | | | Units |
| MacroCTA | NIPAM | Theory | NMR | SEC-TD | Ð | NIPAM |
| MacroCTA-A | 99 | 5290 | 5345 | 5560 | 1.14 | 45 |

### MacroCTA-B (P(NIPAM₅₁-co-DMAEMA₂₉)-S(C═S)SC₄H₉)

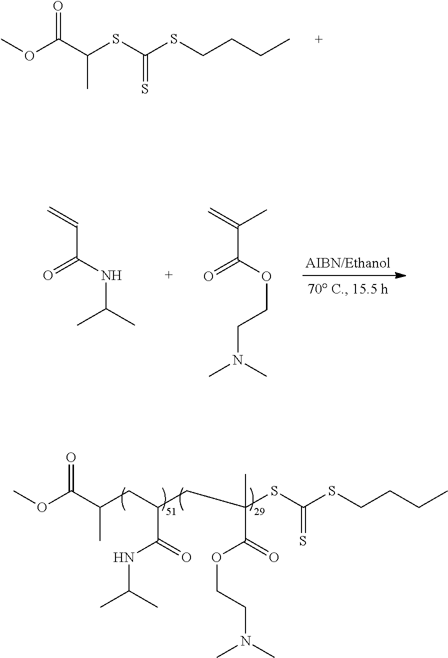

NIPAM (16.15 g, 1.43×10₋₁ mol) and DMAEMA (13.46 g, 8.56×10₋₂ mol) were dissolved in 25.5 mL of ethanol and stirred with basic alumina (100 mg) for 30 min to remove inhibitor. The mixture was filtered, and MCEBTTC RAFT agent (0.72 g. 2.85×10₋₃ mol) and AIBN (0.070 g, 4.26×10₋₄ mol) were added. The solution was degassed with argon for 40 min. Polymerization of MacroCTA-B was carried out at 70° C. for 15.5 h. The reaction was quenched by exposure to air and used directly in the emulsion polymerization step. A small portion of the polymer crude was taken for ¹H NMR characterization (to determine NIPAM and DMAEMA conversion). Another small portion of the polymer crude was taken and purified via dialysis against Milli-Q water (3500 MWCO), freeze dried, then characterized by ₁H NMR and SEC (to determine repeating units and molecular weight distribution of the polymer).

TABLE S2
Synthesis of MacroCTA-B by RAFT-mediated polymerization.

| | Conv. (%) | | Mₙ | | | | Repeating Units | |
|---|---|---|---|---|---|---|---|---|
| MacroCTA | NIPAM | DMAEMA | Theory | ¹H NMR | SEC-TD | Ð | NIPAM | DMAEMA |
| MacroCTA-B | 94 | 100 | 10290 | 10580 | 12000 | 1.24 | 51 | 29 |

Synthesis of multifunctional nanoworms (NWs)—Synthesis of base nanoworms (NWbase) (FIG. 1A)—To the reactor under an argon blanket, MacroCTA-A/EtOH solution (27.1028 g, 2.02×10₋₃ mol), MacroCTA-B/EtOH solution (30.1925 g, 1.58×10₋₃ mol), SDS (1.1229 g, 3.89×10₋₃ mol) and 460 mL of cold Milli-Q H₂O were added. The solution was stirred in a 0° C. ice bath at 250 rpm until all components dissolved. The stirring rate was decreased to 50 rpm. AIBN (0.0887 g, 5.40×10₋₄ mol) was dissolved in STY (20.9936 g, 2.02×10₋₁ mol) and the solution added to the reaction under argon blanket. The reaction solution was degassed by bubbling with argon for 120 min. The stirring rate was increased to 250 rpm, then the reactor was heated to 70° C. in a temperature-controlled oil bath and the emulsion polymerization was allowed to proceed for 5 h. At the 4 h mark, approx. 0.1 mL of the polymer latex was taken and characterized by ₁H NMR (to determine conversion of STY). After 5 h, the reaction was quenched by exposing reactor to air. A sample was taken, freeze dried, and later characterized by ¹H NMR and SEC (to determine final conversion and Mₙ). The volatiles were removed by applying a gentle compressed air flow while stirring at 70° C. for 2 h. A sample was taken and characterized by DLS (to determine particle size). To transform the polymer latex to nanoworms, the stirring rate was decreased to 50 rpm and toluene (8.6750 g, 20 μL/mL) was added. The reactor was then removed from heating and allowed to cool to ambient temperature, this took ˜24 h.

TABLE S3
Synthesis of NW_{base} by RAFT-mediated emulsion polymerization at 70°
C. in water for 5 h using SDS as a surfactant and AIBN as an initiator.

| Emulsion Conditions | | | | Conv. (%) | | | | DLS | |
|---|---|---|---|---|---|---|---|---|---|
| Scale | AIBN | Temp. | Time | ¹H NMR | Mₙ | | | Dₕ | |
| (mL) | (eg.) | (° C.) | (h) | (4 h) | ¹H NMR | SEC-TD | Ð | (nm) | PDI |
| 500 | 0.15 | 70 | 5 | 85 | 12720 | 12610 | 1.37 | 1365 | 0.501 |

Removal of toluene from latex by rotary evaporation—Toluene was removed from the latex dispersion (500 mL) via rotary evaporation at 22° C. (25-50 mbar pressure) for ˜3 h. Nanoworms kept their morphology.

Quaternization of NWbase with iodooctane and propargyl bromide (QPO-NWbase) (FIG. 1C)—Small amount (0.4 mL) of NWbase in water was freeze-dried (three repeats) to determine wt. % of NWbase. It was found that wt. % of NWbase=7.5 wt. %. Based on this determined wt. % of NWbase, calculations were performed to figure out the amounts of iodooctane, propargyl bromide and DMSO to be added. 36 mL of Milli-Q water was added to NWbase aqueous dispersion (34.23 g of NWbase in 420 mL of Milli-Q water). 1.0497 g of iodooctane in 56.3 mL of DMSO was added to NWbase aqueous dispersion and the reaction mixture was stirred in a 1 L glass Schott bottle for 15 h at 23° C. After that 2.5 g of propargyl bromide (80 wt. % in toluene) in 24.14 mL of DMSO was added to the NWbase reaction mixture and the reaction was carried out for extra 12 h at 23° C. Then, the reaction mixture was dialyzed (3500 MWCO) against Milli-Q water in a 5 L beaker for 24 h (changed Milli-Q water four times, approximately every 6 h) to remove unreacted iodooctane and propargyl bromide. After dialysis was completed, the product QPO-NWbase was transferred from dialysis bags to a 1 L Schott bottle for further reaction. Small amount of QPO-NWbase was freeze-dried (3 Eppendorf tubes with 0.4 mL each) to calculate wt. % of solid in final dispersion.

TABLE S4
Synthesis of QPO-NW_{base}.

| Quaternization conditions | | | | Characterizations | | | |
|---|---|---|---|---|---|---|---|
| | | | Propargyl | | Zeta- | % of | |
| Scale | DMAEMA | Iodooctane | bromide | Time | potential | quaternization | |
| (g) | (eq.) | (eq.) | (eq.) | (h) | (mV) | (¹H NMR) | TEM |
| 34 | 1 | 0.13 | 0.5 | 27 | +52 | 93 | worms |

Synthesis of NW_{S, O, G, C} (FIG. 1D)—Small amount (0.4 mL) of QPO-NW_{base} in water was freeze-dried (three repeats) to determine wt. % of QPO-NW_{base}. It was found that wt. % of QPO-NW_{base}=3.9 wt. %. Based on this determined wt. % of QPO-NW_{base}, calculations were performed to figure out the amounts of guanidine azide, polygalactose azide, coumarin azide, copper sulfate, ascorbic acid and DMSO to be added. 614 mL of 3.9 wt. % QPO-NW_{base} dispersion (contains 25 g of QPO-NW_{base} solid) were placed in a 2 L two neck round bottom flask. Then, the following solutions were added to the flask: (i) guanidine azide (1.13 g) in 39.6 mL of DMSO, (ii) 3-azido-7-hydroxycoumarin (0.1796 g) in 39.6 mL of DMSO, (iii) polygalactose azide (2.98 g) in 39.6 mL of DMSO. The resulting dispersion was purged with argon for 6 h. After that, ascorbic acid (10.89 g) in degassed Milli-Q water (35 mL, purged with argon for 2 h) was injected to the reaction mixture in the flask via a degassed syringe followed by the injection of copper sulfate (4.23 g) in degassed Milli-Q water (25 mL, purged with argon for 2 h). The reaction was carried out for 19 h at 23° C. under argon atmosphere in the dark. The reaction was stopped by exposure to the air and 7 g of aluminum basic oxide was added in portion (0.5 g) and stirred for 2 h. After that the stirring was stopped and aluminum oxide particles settled down to the bottom of flask and nanoworms dispersion was decanted to a 1 L bottle. The nanoworms dispersion was dialyzed against Milli-Q water (3500 MWCO, changed water every 3 h, dialysis bags were changed to new ones after 8 h) in a 3 L beaker for 17 h. The resulting solution was freeze-dried to obtain NW_{S, O, G, C} as a light yellowish powder. Zeta-potential (2 mg/mL, 25° C., Milli-Q water)=+26.5 mV.

Coating and droplet spreading of armrest surface—A dispersion of NWS, O, G, C at 1.5 wt. % in 70% autoclave water and 30% ethanol was prepared. An armrest (1 cm×1 cm) was cleaned by wiping with 100% ethanol followed by wiping with Milli-Q water and drying at ambient temperature. NWS, O, G, C dispersion was sprayed onto an armrest at the distance approximately 7 cm from an armrest. The armrest was dried completely by gently blowing with an air dryer. The spraying-drying cycle was repeated four times. A 10 μL solution of either 0.2 M PBS solution at pH 6.5 or pH 7.4 and 23° C. was placed on an uncoated armrest (1 cm×1 cm) or on a NWS, O, G, C-coated armrest (1 cm×1 cm) at 23° C. Photographs of droplet spreading were taken at 5 and 30 min after the droplet addition.

Surface testing for virucidal activity against SARS-CoV-2: All SARS-CoV-2 infection cultures were conducted within the high containment facilities in a PC3 laboratory at the Doherty Institute. Stocks of SARS-CoV-2 isolate hCoV-19/Australia/VIC17991/2020 (B.1.1.7 variant) and VIC18440 (Indian-2, delta variant) were produced from infected Vero cell supernatants. The genomic sequence of each stock of SARS-CoV-2 isolate was confirmed to match the publicly available data of the original virus isolate (www.gisaid.org, accession numbers: EPI_ISL_779606 and EPI_ISL_1913206, respectively). Virus inoculum was created by adding an equal volume of virus stocks to filtered sterilized Sorensen's pH buffer at pH=6.5. Then, 50 μL of inoculum was added dropwise to the surface of each material and allowed to incubate at room temperature for the duration specified. The surface of each material was then washed 8 times by pipetting with 500 μL infection media, with care taken not to scratch the surface. Eluate was then collected and infectious virus titer quantified via a 50% Tissue Culture Infectious Dose (TCID50) assay using the appropriate cell line (Vero cells for SARS-CoV-2). Viral RNA was also extracted from the eluate using the QiaAmp Viral RNA extraction kit (Qiagen, Australia) as per the manufacturer's instructions and stored at −80° C. To evaluate the amount of virus genome present in each sample, we performed a quantitative reverse-transcription PCR (qRT-PCR) for detection of the SARS-CoV-2 envelope (E) gene and influenza nucleoprotein (NP) gene segment. Using the SuperScriptIII OneStep RT-PCR System with Platinum® Taq DNA Polymerase (Invitrogen, Carlsband, CA, USA), the RT-qPCR assay comprised of 5 μL RNA, 12.5 μL 2× Reaction Master Mix, 0.4 μL of 50 mM MgSO4, 1 μL Superscript III/Taq Enzyme Mix, 0.4 μM forward and reverse primers, and 0.2 μM primer probe (using previously published primer-probe sequences49), 1 μl of 1 mg/mL Bovine Serum Albumin (BSA) and 2.6 μL RNAse free H2O. The RT-qPCR assay was performed on a Bio-Rad CFX96 Touch Real-Time PCR Detection System (Bio-Rad, Hercules, CA, USA) using the following conditions: a denaturation step at 55° C. for 10 min and 95° C. for 3 min, followed by 45 cycles of amplification (94° C. for 15 s and 58° C. for 30 s). A known amount of influenza A virus and SARS-CoV-2 RNA (generated previously from virus stock cultures) diluted two-fold was used to generate a standard curve. The Ct values from the standard curve were used to interpolate the amount of SARS-CoV-2 RNA in each of the samples.

Surface Exposure Assay—SARS-CoV-2 inoculum was created by adding an equal volume of virus stocks to of filter sterilized Sorensen's pH buffer at pH=6.5. 50 μL of inoculum was then added dropwise to the surface of each material, then allowed to incubate at room temperature for the duration specified. The surface of each material was then washed 8 times by pipetting with 500 μL infection media, with care taken not to scratch the surface. Eluate was then collected and infectious virus titre quantified via a 50% Tissue Culture Infectious Dose (TCID50) assay. Viral RNA was also extracted from the eluate using the QiaAmp Viral RNA extraction kit (Qiagen, Australia) as per the manufacturer's instructions and stored at −80° C. To evaluate the amount of virus genome present in each sample, we performed a reverse-transcription quantitative PCR (RT-qPCR) for detection of the SARS-CoV-2 envelope (E) gene. Using the SuperScriptIII OneStep RT-PCR System with Platinum® Taq DNA Polymerase (Invitrogen, Carlsband, CA, USA), the RT-qPCR assay comprised of 5 L RNA, 12.5 L 2× Reaction Master Mix, 0.4 μL of 50 mM MgSO4, 1 μL Superscript III/Taq Enzyme Mix, 0.4 μM forward (5′-ACAGGTACGTTAATAGTTAATAGCGT-3′), 0.4 μM reverse (5′-ATATTGCAGCAGTACGCACACA-3′) primers and 0.2 μM primer probe (5′-FAM-ACACTAGCCATCCTTACTGCGCTTCG-BBQ-3′), 1 μl of 1 mg/mL Bovine Serum Albumin (BSA) and 2.6 μL RNAse free H2O. The RT-qPCR assay was performed on a Bio-Rad CFX96 Touch Real-Time PCR Detection System (Bio-Rad, Hercules, CA, USA) using the following conditions: a denaturation step at 55° C. for 10 min and 95° C. for 3 min, followed by 45 cycles of amplification (94° C. for 15 s and 58° C. for 30 s). A known amount of SARS-CoV-2 RNA (generated previously from virus stock cultures) diluted two-fold was used to generate a standard curve. The Ct values from the standard curve were used to interpolate the amount of SARS-CoV-2 RNA in each of the samples.

Statistical Analysis: All data are graphed as the mean±standard deviation (SD) using GraphPad Prism (v8.4). Data from at least two independent experiments were pooled and graphed unless otherwise stated.

### Test Results of the Coating

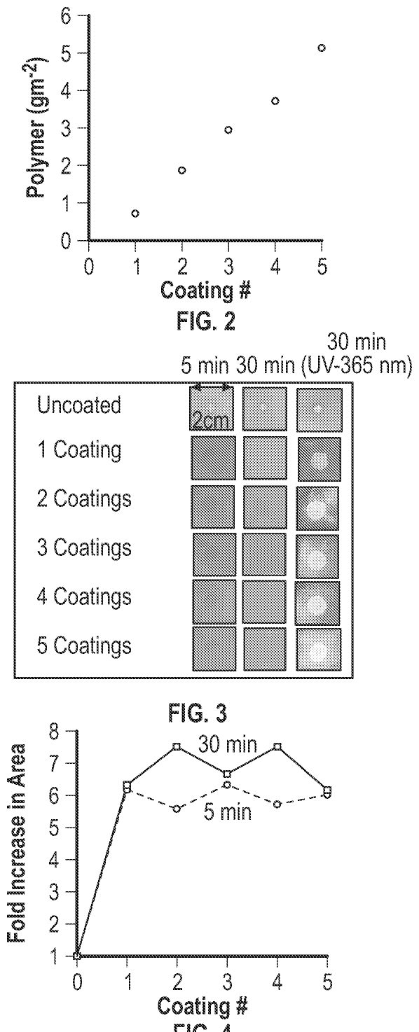

FIG. 2 is a graph depicting the polymer per surface area with increasing amounts of coatings. As can be seen, with an increase in the number of sprays, the amount of polymer increased substantially linearly from 1 g/m² to 5 g/m². The emulsion polymerization was carried out at a 500 mL scale to produce approximately 40 g of polymer. The surface coverage can range from about 8 m² to about 40 m² with a decrease in the number of coatings from 5 to 1, respectively. Placing a water droplet at pH 6.5 on the coated surface (˜2×2 cm) showed rapid spreading.

FIG. 3 is a graph depicting a visualization of the spreading of water droplet at pH 6.5 on surfaces, including noncoated surface, and 1 to 5 coatings. The visualization uses fluorescent light at a wavelength of 365 nm to view the water spread. FIG. 4 is a graph depicting the relative increase in droplet spreading with coating number. The spreading increases approximately 6-fold compared to the uncoated surface, in which relatively no change in spreading was observed with increasing number of sprays after both 5 min and 30 min.

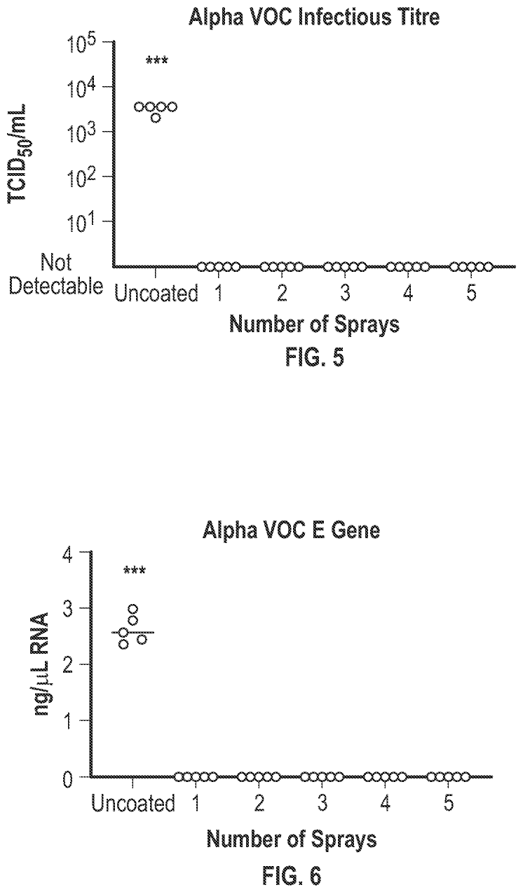

The amount of nanoworm (NW_{S,O,C,G}) on the treated surface was reduced and tested to determine efficacy of virucidal activity against the SARS-CoV-2 (e.g., alpha variant) using the clinical human/Victoria/17991/2020 isolate. Sterile hard plastic surfaces were coated with 1 to 5 sprays of the nanoworms, then virus inoculum (50 μL) prepared in Sorrenson's buffer pH=6.5 was added dropwise onto the nanoworm-coated surface and incubated at room temperature for 30 min. Each surface was then washed with 10-fold media, and the eluate collected and sampled immediately for viral genome extraction and for presence of SARS-CoV-2 infectious titer via performing a 50% tissue culture infectious dose (TCID₅₀) assay. It was discovered that no detectable amount of infectious virus was observed, even after subsequent re-passaging in Vero cells to rescue any residual infectious virus. FIG. 5 depicts a graphical representation relating number of sprays and detectable amount of SARS-CoV-2. Nanoworm-coated plastic hard surfaces or uncoated surfaces were exposed to SARS-CoV-2 human isolate Victoria/17991/2020 (Alpha variant) for 30 min, then washed and eluate assayed for infectious SARS-CoV-2 titer by TCID₅₀ depicted in FIG. 5, or presence of intact SARS-CoV-2 E gene via quantitative reverse transcription polymerase chain reaction (RT-PCR), depicted in the graphical representation of FIG. 6.

One spray (e.g., 1 coating=about 1 g/m²) on the surface was required to eliminate the presence of infectious virus within 30 min of exposure, demonstrating the effectiveness of the anti-viral polymer coating. To support the complete virucidal action of the nanoworm polymer, a quantitative RT-PCR was performed for the presence of an intact SARS-CoV-2 E gene and a significant reduction of virus genome was detected regardless of the number of nanoworm sprays on the treated surface (e.g., FIG. 6).

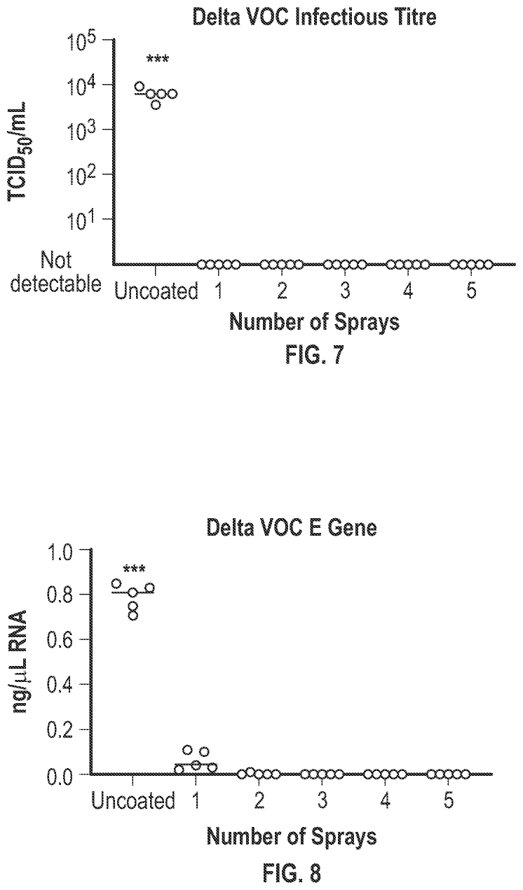

The nanoworm coating was tested against the human/Victoria/18440/2021 isolate with the identical genome to the publicly available data of the original delta (B.1.617.2) virus isolate. Upon examination of the predicted Spike protein glycan array of the delta variant and comparison to the ancestral virus, the mutations in the Spike protein were not expected to significantly change the glycosylation levels. FIG. 7 is a graph illustrating the presence of virucidal activity of nanoworm coated surfaces against SARS-CoV-2 delta variant. Nanoworm-coated plastic hard surfaces or uncoated surfaces were exposed to SARS-CoV-2 human/Victoria/18440/2021 isolate with the identical genome to the delta (B.1.617.2) VOC for 30 min, then washed and eluate assayed for infectious SARS-CoV-2 titer by TCID₅₀. The concentations are shown in FIG. 7. FIG. 8 depicts the presence of intact SARS-CoV-2 E gene via quantitative RT-PCR.

Similar to the results obtained for the alpha variant, the amount of infectious delta virus remaining in the eluate was below detection limit, even after a second passage in the Vero cells. As can be seen, a single spray rendered the virus non-infectious, confirming the efficacy of the coating against SARS-CoV-2 variants, such as the delta variant. Complete inactivation of the delta variant was further supported with the significant reduction of intact viral SARS-CoV-2 E gene present in the sampled eluates shown in FIG. 8.

In comparison to the ancestral SARS-CoV-2 isolate, the omicron SARS-CoV-2 variant has a total of 60 mutations, with 37 (6 deletions, 1 insertion and 30 substitutions) located in the Spike protein. The omicron variant has been reported to share 10 and 5 common mutations with alpha and beta variants, respectively, while 7 mutations each were shared with gamma and delta variants. Compared to the ancestral virus and delta VOC, the omicron Spike protein is less efficiently cleaved and is less fusogenic, indicating changed binding affinity and potential for enzymatic cleavage. Many of the Spike mutations are within the important N-terminal domain, receptor binding domain and receptor binding motif, raising concerns about enhanced transmission and immune evasion.

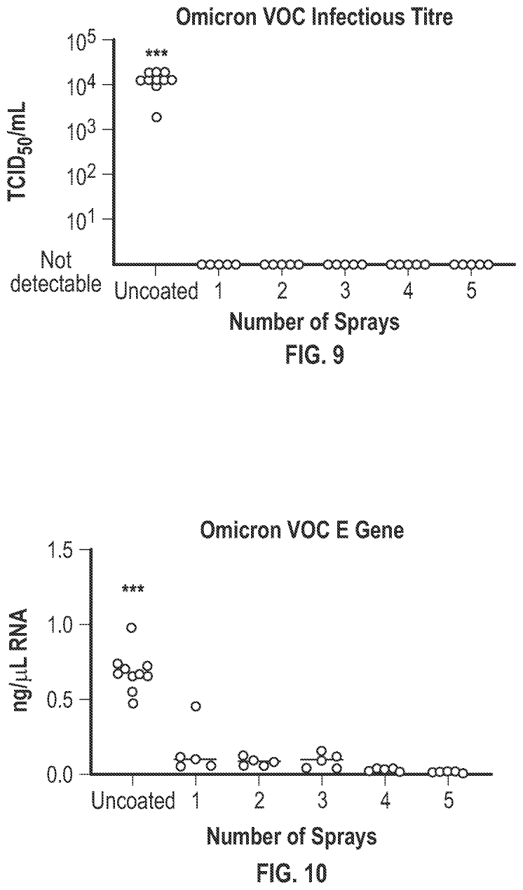

Assays were prepared using the omicron variant (human/NSW/1933/2021; www.gisaid.org, accession number: EPI_ISL_3007291) exposed to surfaces treated with 1 to 5 sprays of the nanoworm coating. Nanoworm-coated plastic hard surfaces or uncoated surfaces were exposed to SARS-CoV-2 human/NSW/1933/2021 isolate with the identical genome to the omicron (B.1.1.529; BA.1 lineage) VOC for 30 min, then washed and eluate assayed for infectious SARS-CoV-2 titer by TCID₅₀ (e.g., shown in FIG. 9) and for presence of intact SARS-CoV-2 E gene via quantitative RT-PCR (e.g., shown in FIG. 10).

As can be seen, a single spray of coating on the surface was sufficient to render the virus non-infectious, as the eluate was found to be below detection limit even after a second passage in the Vero cells and there was a significant reduction in the amount of detectable intact viral SARS-CoV-2 E gene. These data further support that the polygalactose targets binding to the glycosylated regions of the viral particles independent of the mutations presented on the spike.

The polymer nanoworms were tested for both skin sensitivity on mice and oral ingestion toxicity in rats using ethically approved animal models to evaluate the potential safe use on masks and high-touch surfaces. These studies were carried out by an independent agent in compliance with OECD Principle of Good Laboratory Practice. The skin sensitivity potential of NW_{S,O,C,G} was determined by administering 25 μL polymer as a topical application onto the dorsum of each ear of female CBA/CaH mice over a 6 day period. On day 6, 20 μCi of H-thymidine was injected intravenously, and the auricular lymph nodes dissected 5 hours later. The stimulation index (SI) for the positive control (α-hexylcinnamaldehyde) was found to be 12.7, while the SI for the polymer nanoworms at 2.5, 5 and 10 w/v % was 1.08, 1.24 and 1.42, respectively. An SI value greater than 3 represents a potential sensitizing agent. The non-clinical acute toxicity of NW_{S,O,C,G} was determined through a single bolus dose, ranging from 10 to 1000 mg/kg, in female Sprague Dawley rats. Morbidity and mortality was observed daily during the acclimation period and twice daily after ingestion of the polymer over a 15 day period. There was no finding of either morbidity or mortality, with no changes in body weight or macroscopic pathology findings, supporting that the oral ingestion of the polymer was well tolerated by the rats even with high doses of 1000 mg/kg. These combined data support that the polymer nanoworms were non-toxic when ingested and did not cause irritation to the skin, strongly suggesting that this polymer is safe to use in syrface applications, such as face coverings.

### Additional Aspects

Clause A1. A method of disposing a nanostructure onto a surface, comprising: disposing a single layer of a solution or emulsion comprising the nanostructure on the surface, the surface comprising a SARS-CoV-2 virus (e.g., BA.1) disposed thereon, the nanostructure comprising a compound or salt thereof, the compound comprising: one or more styrene units; one or more N-alkylacrylamide units; a moiety represented by the formula:

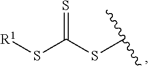

wherein R¹ is alkyl; a moiety represented by the formula:

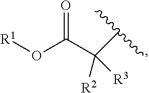

where R¹ is alkyl (such as methyl, ethyl, propyl, butyl) and R² and R³ are independently hydrogen or alkyl (such as methyl, ethyl, propyl, butyl); and a plurality of N,N-(dialkylamino)(divalent alkyl) alkylacrylate units, wherein: one or more of the N,N-(dialkylamino)(divalent alkyl)alkylacrylate units has an unsubstituted nitrogen, one or more of the N,N-(dialkylamino)(divalent alkyl)alkylacrylate units is substituted with a C₁-C₁₆ alkyl moiety (such as a C₄-C₁₂ alkyl moiety, such as an octane moiety), and one or more of the N,N-(dialkylamino)(divalent alkyl) alkylacrylate units is substituted with a moiety selected from the group consisting of guanidine, polygalactose, coumarin, and combination(s) thereof.

Clause A2. The method of Clause A1, wherein the SARS-CoV-2 virus is B.1.1.529 SARS-CoV-2 virus.

Clause A3. The method of Clauses A1 or A2, wherein the surface is a surface of an item of personal protective equipment.

Clause A4. The method of any of Clauses A1 to A3, wherein the surface is an interior or exterior surface of an aircraft, a ship, a train, a terminal, or a spacecraft.

Clause A5. The method of any of Clauses A1 to A4, wherein the emulsion or solution has a concentration of the nanostructure of about 0.5 wt % to about 3 wt %.

Clause A6. The method of any of Clauses A1 to A5, wherein the nanostructure is a nanoworm.

Clause A7. The method of any of Clauses A1 to A6, wherein the nanostructure is a nanorod.

Clause A8. The method of any of Clauses A1 to A7, wherein the compound consists of: one or more styrene units; one or more N-alkylacrylamide units; a moiety represented by the formula:

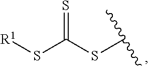

wherein R¹ is alkyl; a moiety represented by the formula:

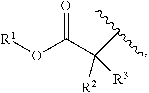

where R¹ is alkyl (such as methyl, ethyl, propyl, butyl) and R² and R³ are independently hydrogen or alkyl (such as methyl, ethyl, propyl, butyl); and a plurality of N,N-(dialkylamino)(divalent alkyl) alkylacrylate units, wherein: one or more of the N,N-(dialkylamino)(divalent alkyl)alkylacrylate units has an unsubstituted nitrogen, one or more of the N,N-(dialkylamino)(divalent alkyl)alkylacrylate units is substituted with a C₁-C₁₆ alkyl moiety (such as a C₄-C₁₂ alkyl moiety, such as an octane moiety), and one or more of the N,N-(dialkylamino)(divalent alkyl) alkylacrylate units is substituted with a moiety selected from the group consisting of guanidine, polygalactose, coumarin, and combination(s) thereof.

Clause A9. A method for treating a condition comprising administering to a subject a therapeutically effective amount of a nanostructure comprising a compound, or a pharmaceutically acceptable salt thereof, wherein the condition includes a viral infection as a result of a SARS-CoV-2 virus (e.g., BA.1), wherein the compound comprises: one or more styrene units; one or more N-alkylacrylamide units; a moiety represented by the formula:

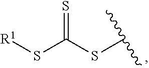

wherein R¹ is alkyl; a moiety represented by the formula:

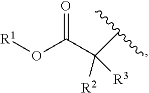

where R¹ is alkyl (such as methyl, ethyl, propyl, butyl) and R² and R³ are independently hydrogen or alkyl (such as methyl, ethyl, propyl, butyl); and a plurality of N,N-(dialkylamino)(divalent alkyl)alkylacrylate units, wherein: one or more of the N,N-(dialkylamino)(divalent alkyl)alkylacrylate units has an unsubstituted nitrogen, one or more of the N,N-(dialkylamino)(divalent alkyl)alkylacrylate units is substituted with a C₁-C₁₆ alkyl moiety (such as a C₄-C₁₂ alkyl moiety, such as an octane moiety), and one or more of the N,N-(dialkylamino)(divalent alkyl)alkylacrylate units is substituted with a moiety selected from the group consisting of guanidine, polygalactose, coumarin, and combination(s) thereof.

Clause A10. The method of Clause A9, wherein the SARS-CoV-2 virus is a B.1.1.529 SARS-CoV-2 virus.

Clause A11. The method of Clauses A9 or A10, wherein the nanostructure is a nanoworm.

Clause A12. The method of any of Clauses A9 to A11, wherein the nanostructure is a nanorod.

Clause A13. The method of any of Clauses A9 to A12, wherein the compound consists of: one or more styrene units; one or more N-alkylacrylamide units; a moiety represented by the formula:

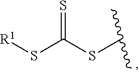

wherein R¹ is alkyl; a moiety represented by the formula:

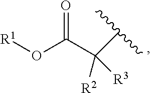

where R¹ is alkyl (such as methyl, ethyl, propyl, butyl) and R² and R³ are independently hydrogen or alkyl (such as methyl, ethyl, propyl, butyl); and a plurality of N,N-(dialkylamino)(divalent alkyl) alkylacrylate units, wherein: one or more of the N,N-(dialkylamino)(divalent alkyl)alkylacrylate units has an unsubstituted nitrogen, one or more of the N,N-(dialkylamino)(divalent alkyl)alkylacrylate units is substituted with a C₁-C₁₆ alkyl moiety (such as a C₄-C₁₂ alkyl moiety, such as an octane moiety), and one or more of the N,N-(dialkylamino)(divalent alkyl) alkylacrylate units is substituted with a moiety selected from the group consisting of guanidine, polygalactose, coumarin, and combination(s) thereof.

Clause B1. A method of disposing a nanostructure onto a surface, consisting of:
disposing a single layer of a solution or emulsion comprising the nanostructure on the surface, the nanostructure comprising a compound or salt thereof, the compound comprising:
   one or more (substituted or unsubstituted) styrene units;
   one or more N-alkylacrylamide units;
   a moiety represented by the formula:

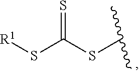

wherein R¹ is alkyl (branched or linear, substituted or unsubstituted);
   a moiety represented by the formula:

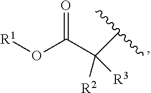

R² where R¹ is alkyl (such as methyl, ethyl, propyl, butyl) and R² and R³ are independently hydrogen or alkyl (such as methyl, ethyl, propyl, butyl); and
a plurality of N,N-(dialkylamino)(divalent alkyl)alkylacrylate units, wherein:
   one or more of the N,N-(dialkylamino)(divalent alkyl)alkylacrylate units has an unsubstituted nitrogen,
   one or more of the N,N-(dialkylamino)(divalent alkyl)alkylacrylate units is substituted with a C₁-C₁₆ alkyl moiety (such as a C₄-C₁₂ alkyl moiety, such as an octane moiety), and
   one or more of the N,N-(dialkylamino)(divalent alkyl)alkylacrylate units is substituted with a moiety selected from the group consisting of guanidine, polygalactose, coumarin, and combination(s) thereof.

Clause B2. The method of Clause B1, wherein the surface is a surface of an item of personal protective equipment.

Clause B3. The method of Clauses B1 or B2, wherein the surface is an interior or exterior surface of an aircraft, a ship, a train, a terminal, or a spacecraft. Clause B4. The method of any of any of Clauses B1 to B3, wherein the emulsion or solution has a concentration of the nanostructure of about 0.5 wt % to about 3 wt %.

Clause B5. The method of any of Clauses B1 to B4, wherein the nanostructure is a nanoworm.

Clause B6. The method of any of Clauses B1 to B5, wherein the nanostructure is a nanorod.

Clause B7. A method for treating a condition comprising administering to a subject a therapeutically effective amount of a nanostructure comprising a compound, or a pharmaceutically acceptable salt thereof, wherein the condition includes a viral infection as a result of SARS-CoV-2 virus, wherein the compound comprises: one or more (substituted or unsubstituted) styrene units; one or more N-alkylacrylamide units; a moiety represented by the formula:

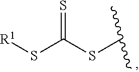

wherein R¹ is alkyl (branched or linear, substituted or unsubstituted); a moiety represented by the formula:

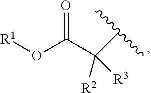

where R¹ is alkyl (such as methyl, ethyl, propyl, butyl) and R² and R³ are independently hydrogen or alkyl (such as methyl, ethyl, propyl, butyl); and a plurality of N,N-(dialkylamino)(divalent alkyl)alkylacrylate units, wherein: one or more of the N,N-(dialkylamino)(divalent alkyl)alkylacrylate units has an unsubstituted nitrogen, one or more of the N,N-(dialkylamino)(divalent alkyl)alkylacrylate units is substituted with a C₁-C₁₆ alkyl moiety (such as a C₄-C₁₂ alkyl moiety, such as an octane moiety), and one or more of the N,N-(dialkylamino)(divalent alkyl)alkylacrylate units is substituted with a moiety selected from the group consisting of guanidine, polygalactose, coumarin, and combination(s) thereof.

Clause B8. The method of Clause B7, wherein the condition is a viral infection as a result of B.1.1.7 SARS-CoV-2 virus or B.1.617.2 SARS-CoV-2 virus.

Clause B9. The method of Clauses B7 or B8, wherein the nanostructure is a nanoworm.

Clause B10. The method of any of Clauses B7 to B9, wherein the nanostructure is a nanorod.

Clause B11. A method of forming a nanostructure, the method comprising: introducing, in a reactor, a styrene monomer with (1) a first polymer having N-alkylacrylamide units and (2) a second polymer having N,N-(dialkylamino)(divalent alkyl)alkylacrylate units and N-alkylacrylamide units to form a mixture, wherein the first polymer is free of N,N-(dialkylamino)(divalent alkyl)alkylacrylate units; and introducing an initiator compound to the mixture to form a second mixture comprising the nanostructure.

Clause B12. The method of Clause B11, wherein the first polymer consists of: the N-alkylacrylamide as N-isopropylacrylamide units, a moiety represented by the formula:

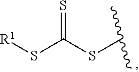

wherein R¹ is alkyl (branched or linear, substituted or unsubstituted), and a moiety represented by the formula:

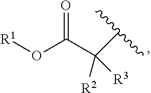

where R¹ is alkyl (such as methyl, ethyl, propyl, butyl) and R² and R³ are independently hydrogen or alkyl (such as methyl, ethyl, propyl, butyl).

Clause B13. The method of claim B1, wherein the second polymer consists of: the N,N-(dialkylamino)(divalent alkyl)alkylacrylate units as N,N-(dimethylamino)ethyl methacrylate units, the N-alkylacrylamide units as N-isopropylacrylamide units, a moiety represented by the formula:

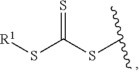

wherein R¹ is alkyl (branched or linear, substituted or unsubstituted), and a moiety represented by the formula:

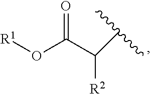

wherein R is alkyl (branched or linear, substituted or unsubstituted) and R² is alkyl (branched or linear, substituted or unsubstituted).

Clause B14. The method of any of Clauses B11 to B13, wherein the initiator compound is a peroxide, a hydroperoxide, or an azo initiator.

Clause B15. The method of any of Clauses B11 to B14, wherein the initiator is azobisisobutyronitrile.

Clause B16. The method of any of Clauses B11 to B15, wherein introducing the styrene monomer with the first polymer and the second polymer is performed at a temperature of about −10° C. to about 10° C.

Clause B17. The method of any of Clauses B11 to B16, further comprising heating the second mixture at a temperature of about 60° C. to about 80° C.

Clause B18. The method of any of Clauses B11 to B17, wherein the method is performed under inert atmosphere and the method further comprises quenching the second mixture by exposing the reactor to air.

Clause B19. The method of any of Clauses B11 to B18, further comprising isolating the nanostructure from the second mixture and introducing an organic solvent with the nanostructure to form a nanoworm.

Clause B20. The method of any of Clauses B11 to B19, wherein the organic solvent is toluene.

Clause B21. The method of any of Clauses B11 to B20, wherein the reactor has a volume of greater than 1 kiloliter.

Clause B22. The method of any of Clauses B11 to B21, wherein the nanostructure is a nanoworm.

Clause B23. The method of any of Clauses B11 to B22, further comprising ultrasonically cutting the nanoworm to form a plurality of nanorods.

Clause C1. A nanoworm comprising a compound, or salt thereof, the compound comprising: one or more N-isopropylacrylamide units; a moiety represented by the formula:

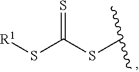

wherein R¹ is alkyl (such as the moiety is represented by the formula:

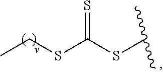

wherein v is an integer of 1 to 20); a moiety represented by the formula:

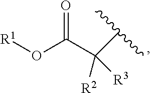

where R¹ is alkyl (such as methyl, ethyl, propyl, butyl) and R² and R³ are independently hydrogen or alkyl (such as methyl, ethyl, propyl, butyl); and a plurality of N,N-(dimethylamino)ethyl methacrylate units, wherein: one or more of the N,N-(dimethylamino)ethyl methacrylate units have an unsubstituted nitrogen, one or more of the N,N-(dimethylamino)ethyl methacrylate units is substituted with a C₁-C₁₆ alkyl moiety (such as a C₄-C₁₂ alkyl moiety, such as an octane moiety), and one or more of the N,N-(dimethylamino)ethyl methacrylate units is substituted with a moiety selected from the group consisting of guanidine, polygalactose, coumarin, and combination(s) thereof.

Clause C2. The nanoworm of Clause C1, the compound further comprising one or more styrene units.

Clause C3. The nanoworm of Clauses C1 or C2, one or more of the N,N-(dimethylamino)ethyl methacrylate units is substituted with a polygalactose.

Clause C4. The nanoworm of any of Clauses C1 to C3, wherein the moiety selected from the group consisting of guanidine, polygalactose, coumarin, and combination(s) thereof is bonded with the corresponding N,N-(dimethylamino)ethyl methacrylate unit via a divalent 5-H triazole.

Clause C5. A mask comprising the nanoworm of any of Clauses C1 to C4 1 disposed on or disposed in the mask.

Clause C6. A method of disposing a nanoworm onto a surface, comprising:
disposing the nanoworm on the surface, the nanoworm comprising a compound, the compound comprising: one or more N-isopropylacrylamide units; a moiety represented by the formula:

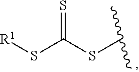

wherein R¹ is alkyl (such as the moiety is represented by the formula:

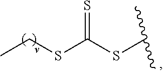

wherein v is an integer of 1 to 20); a moiety represented by the formula

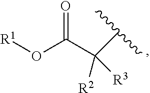

where R¹ is alkyl (such as methyl, ethyl, propyl, butyl) and R² and R³ are independently hydrogen or alkyl (such as methyl, ethyl, propyl, butyl); and a plurality of N,N-(dimethylamino)ethyl methacrylate units, wherein: one or more of the N,N-(dimethylamino)ethyl methacrylate units have an unsubstituted nitrogen, one or more of the N,N-(dimethylamino)ethyl methacrylate units is substituted with a C₁-C₁₆ alkyl moiety (such as a C₄-C₁₂ alkyl moiety, such as an octane moiety), and one or more of the N,N-(dimethylamino)ethyl methacrylate units is substituted with a moiety selected from the group consisting of guanidine, polygalactose, coumarin, and combination(s) thereof.

Clause C7. The method of Clause C6, wherein the surface is a surface of an item of personal protective equipment.

Clause C8. A method for treating a condition comprising administering to a subject a therapeutically effective amount of a nanoworm comprising a compound, or a pharmaceutically acceptable salt thereof, wherein the condition includes viral infections, bacterial infections, chronic inflammatory disorders, acute inflammatory disorders, or cancer, wherein the compound comprises: one or more N-isopropylacrylamide units; a moiety represented by the formula:

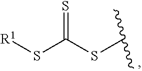

wherein R¹ is alkyl (such as the moiety is represented by the formula:

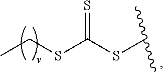

wherein v is an integer of 1 to 20); a moiety represented by the formula:

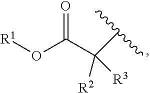

where R¹ is alkyl (such as methyl, ethyl, propyl, butyl) and R² and R³ are independently hydrogen or alkyl (such as methyl, ethyl, propyl, butyl); and a plurality of N,N-(dimethylamino)ethyl methacrylate units, wherein: one or more of the N,N-(dimethylamino)ethyl methacrylate units have an unsubstituted nitrogen, one or more of the N,N-(dimethylamino)ethyl methacrylate units is substituted with a C₁-C₁₆ alkyl moiety (such as a C₄-C₁₂ alkyl moiety, such as an octane moiety), and one or more of the N,N-(dimethylamino)ethyl methacrylate units is substituted with a moiety selected from the group consisting of guanidine, polygalactose, coumarin, and combination(s) thereof.

Clause C9. The method of any of Clauses C1 to C8, wherein the condition is a viral infection as a result of SARS-CoV-2, H1N1, VIC01, B.1.1.7., and combination(s) thereof.

Clause C10. A method of forming a triazole-containing compound, the method comprising: forming the triazole-containing compound by actively or staticly mixing an azide-containing compound with copper iodide and an alkyne-containing compound, the alkyne-containing compound comprising: one or more N-isopropylacrylamide units; a moiety represented by the formula:

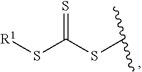

wherein R¹ is alkyl (such as the moiety is represented by the formula:

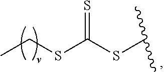

wherein v is an integer of 1 to 20); a moiety represented by the formula:

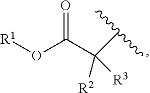

where R¹ is alkyl (such as methyl, ethyl, propyl, butyl) and R² and R³ are independently hydrogen or alkyl (such as methyl, ethyl, propyl, butyl); and a plurality of N,N-(dimethylamino)ethyl methacrylate units, wherein: one or more of the N,N-(dimethylamino)ethyl methacrylate units have an unsubstituted nitrogen, one or more of the N,N-(dimethylamino)ethyl methacrylate units is substituted with a C₁-C₁₆ alkyl moiety (such as a C₄-C₁₂ alkyl moiety, such as an octane moiety), and one or more of the N,N-(dimethylamino)ethyl methacrylate units is substituted with an alkyne moiety, the azide-containing compound selected from the group consisting of azide-containing guanidine, azide-containing polygalactose, azide-containing coumarin (e.g., 3-azido-7-hydroxycoumarin), and combination(s) thereof.

While the foregoing is directed to aspects of the present disclosure, other and further aspects of the disclosure may be devised without departing from the basic scope thereof, and the scope thereof is determined by the claims that follow.

What is claimed is:

1. A method of disposing a nanostructure onto a surface, comprising:
disposing a layer of a solution or emulsion comprising the nanostructure on the surface, the surface comprising a SARS-CoV-2 virus disposed thereon, the nanostructure comprising a compound or salt thereof, the compound comprising:
one or more styrene units;
one or more N-alkylacrylamide units;
a moiety represented by the formula: 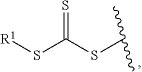
wherein R¹ is alkyl;
a moiety represented by the formula: 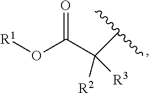
wherein R¹ is alkyl and R² and R³ are independently hydrogen or alkyl; and
a plurality of N,N-(dialkylamino)(divalent alkyl) alkylacrylate units, wherein:
one or more of the N,N-(dialkylamino)(divalent alkyl)alkylacrylate units has an unsubstituted nitrogen,
one or more of the N,N-(dialkylamino)(divalent alkyl)alkylacrylate units is substituted with a C₁-C₁₆ alkyl moiety, and
one or more of the N,N-(dialkylamino)(divalent alkyl) alkylacrylate units is substituted with a moiety selected from the group consisting of guanidine, polygalactose, coumarin, and combination(s) thereof.

2. The method of claim 1, wherein the BA.1 SARS-CoV-2 virus is B.1.1.529 SARS-CoV-2 virus.

3. The method of claim 1, wherein the surface is a surface of an item of personal protective equipment.

4. The method of claim 1, wherein the surface is an interior or exterior surface of an aircraft, a ship, a train, a terminal, or a spacecraft.

5. The method of claim 1, wherein the emulsion or solution has a concentration of the nanostructure of about 0.5 wt % to about 3 wt %.

6. The method of claim 1, wherein the nanostructure is a nanoworm.

7. The method of claim 1, wherein the nanostructure is a nanorod.

8. The method of claim 1, wherein the compound consists of:
one or more styrene units;
one or more N-alkylacrylamide units;
a moiety represented by the formula: 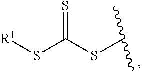
wherein R¹ is alkyl;
a moiety represented by the formula: 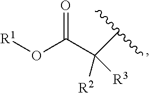
wherein R¹ is alkyl and R² and R³ are independently hydrogen or alkyl; and
a plurality of N,N-(dialkylamino)(divalent alkyl) alkylacrylate units, wherein:
one or more of the N,N-(dialkylamino)(divalent alkyl)alkylacrylate units has an unsubstituted nitrogen,
one or more of the N,N-(dialkylamino)(divalent alkyl)alkylacrylate units is substituted with a C₁-C₁₆ alkyl moiety, and
one or more of the N,N-(dialkylamino)(divalent alkyl) alkylacrylate units is substituted with a moiety selected from the group consisting of guanidine, polygalactose, coumarin, and combination(s) thereof.

9. A method of disposing a nanostructure onto a surface, comprising:
disposing the nanostructure on the surface, the nanostructure comprising a compound, the compound comprising:
one or more N-isopropylacrylamide units;
a moiety represented by the formula: 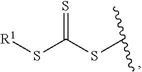
wherein R¹ is alkyl;
a moiety represented by the formula: 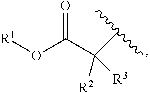
wherein R¹ is alkyl and R² and R³ are independently hydrogen or alkyl; and
a plurality of N,N-(dimethylamino)ethyl methacrylate units, wherein:
one or more of the N,N-(dimethylamino)ethyl methacrylate units have an unsubstituted nitrogen,
one or more of the N,N-(dimethylamino)ethyl methacrylate units is substituted with a C₁-C₁₆ alkyl moiety, and
one or more of the N,N-(dimethylamino)ethyl methacrylate units is substituted with a moiety selected from the group consisting of guanidine, polygalactose, coumarin, and combination(s) thereof.

10. The method of claim 9, wherein the surface is a surface of an item of personal protective equipment.

11. The method of claim 9, wherein the surface is an interior or exterior surface of an aircraft, a ship, a train, a terminal, or a spacecraft.

12. The method of claim 9, wherein the emulsion or solution has a concentration of the nanostructure of about 0.5 wt % to about 3 wt %.

13. The method of claim 9, wherein the nanostructure is a nanoworm.

14. The method of claim 9, wherein the nanostructure is a nanorod.

15. A nanostructure comprising a compound, or salt thereof, the compound comprising:
one or more N-isopropylacrylamide units;
a moiety represented by the formula: 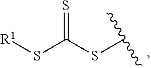
wherein R¹ is alkyl;
a moiety represented by the formula: 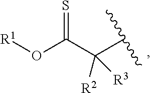
wherein R¹ is alkyl and R² and R³ are independently hydrogen or alkyl; and
a plurality of N,N-(dimethylamino)ethyl methacrylate units, wherein:
one or more of the N,N-(dimethylamino)ethyl methacrylate units have an unsubstituted nitrogen,
one or more of the N,N-(dimethylamino)ethyl methacrylate units is substituted with a C₁-C₁₆ alkyl moiety, and
one or more of the N,N-(dimethylamino)ethyl methacrylate units is substituted with a moiety selected from the group consisting of guanidine, polygalactose, coumarin, and combination(s) thereof.

16. The nanostructure of claim 15, wherein the C₁-C₁₆ alkyl moiety is octane.

17. The nanostructure of claim 15, the compound further comprising one or more styrene units.

18. The nanostructure of claim 15, wherein one or more of the N,N-(dimethylamino)ethyl methacrylate units is substituted with a polygalactose.

19. The nanostructure of claim 15, wherein the moiety selected from the group consisting of guanidine, polygalactose, coumarin, and combination(s) thereof is bonded with the corresponding N,N-(dimethylamino)ethyl methacrylate unit via a divalent 5-H triazole.

20. An item of personal protective equipment comprising the nanostructure of claim 15 disposed on a surface of the mask.
